(19) United States
(12) Patent Application Publication
(10) Pub. No.: US 2026/0174807 A1
(43) Pub. Date: Jun. 25, 2026

(54) PRODUCT FOR PREVENTING AND/OR TREATING OBESITY AND METABOLIC SYNDROME, METHOD OF PREPARATION AND USE THEREOF

(71) Applicant: Minsheng Zhongke Jiayi (Shandong) Biotechnology Co., Ltd., Weifang (CN)

(72) Inventors: Tingtao CHEN, Weifang (CN); Xiaolong HAN, Weifang (CN); Shufeng SI, Weifang (CN); Weichao CAO, Weifang (CN); Jianliang HOU, Weifang (CN); Liyuan Wang, Weifang (CN); Lu QIE, Weifang (CN)

(21) Appl. No.: 18/832,215

(22) PCT Filed: Feb. 18, 2024
(86) PCT No.: PCT/CN2024/077344

§ 371 Date: Jul. 23, 2024

(30) Foreign Application Priority Data
Feb. 27, 2023 (CN) 202310171193.6

(51) Int. Cl. A61K 35/747; A61P 3/00; A61P 3/04; A61P 3/10

(52) U.S. Cl. CPC A61K 35/747; A61P 3/00; A61P 3/04; A61P 3/10

(57) ABSTRACT

The present disclosure relates to the field of microbial therapy, and specifically to a product for preventing and/or treating obesity and a metabolic syndrome. The product includes a Lactobacillus acidophilus strain JYLA-126 and a coating solution thereof. A bacterial solution of the L. acidophilus strain JYLA-126 is coated with a bacterial coating solution to better transport the bacterial solution to the intestinal tract to exert therapeutic effects. The L. acidophilus strain JYLA-126 coating solution is used for intragastric treatment of obesity mice induced by high-fat diet. Various indicators show that the L. acidophilus strain JYLA-126 coating solution may effectively regulate intestinal microflora and maintain intestinal health. The coating solution improves liver glucose metabolism, reduces liver oxidative stress, improves liver lipid metabolism, and alleviates a liver inflammation level. This coating solution improves white adipose accumulation and promotes brown adipose thermogenesis, thereby achieving effective treatment of obesity and metabolic syndrome in mice.

### CROSS REFERENCE TO RELATED APPLICATION

This patent application is a national stage application of the International Patent Application No. PCT/CN2024/077344, filed on Feb. 18, 2024, which claims the benefit and priority of Chinese Patent Application No. 202310171193.6 filed with the China National Intellectual Property Administration on Feb. 27, 2023, both of which are incorporated by reference herein in its entirety as part of the present application.

### REFERENCE TO SEQUENCE LISTING

A computer readable XML file entitled “GWPCTP20240100724_seqlist.xml”, which was created on Jun. 12, 2024, with a file size of about 3,447 bytes, contains the sequence listing for this application, has been filed with this application, and is hereby incorporated by reference in its entirety.

### TECHNICAL FIELD

The present disclosure relates to the technical field of microbial therapy, and specifically relates to a product for preventing and/or treating obesity and a metabolic syndrome, and a method of preparation and use thereof.

### BACKGROUND

Obesity, a chronic metabolic disease caused by multiple factors, is characterized by an increase in the volume and number of fat cells in the body, resulting in an abnormal increase in the percentage of body fat in body weight and excessive deposition of fat in certain body parts. Being overweight or obesity increases the risk of breast cancer, coronary heart disease, type 2 diabetes mellitus, gallbladder disease, osteoarthritis, colon cancer, high blood pressure, and stroke.

With the development of science and technology, intestinal microflora, as an important “organ” that has been ignored for a long time, is receiving an increased attention. The intestinal microflora has been identified as a key factor that affects metabolic health through interactions with diet and intestinal immunity. Alterations in the intestinal microflora and the leakage of its constituents (cells, proteins, and metabolites) across the intestinal barrier in the presence of an unhealthy diet can lead to obesity-related low-grade inflammations and insulin resistance in metabolic tissues. Moreover, people's lifestyle, hygiene habits, and medications may affect the type, quantity, and function of intestinal microorganisms, thereby promoting the occurrence and development of diseases such as metabolic syndrome, obesity, diabetes, and even cancer. Accordingly, studying the interaction between intestinal microflora and metabolism is crucial for host health. However, it is unclear to what extent these changes in intestinal microflora contribute to the metabolism. According to research, Firmicutes has a positive regulatory effect on the decomposition of host proteins, the digestion and absorption of starch, and the synthesis of short-chain fatty acids. Bacteroidota can degrade complex carbon-containing organic matter and generate small-molecule organic acids such as acetic acid and propionic acid to improve host metabolism and play an important role in reducing fat and weight loss. An increase in the fungi/bacteria (F/B) ratio is related to obesity, while an increase in the relative abundance of Firmicutes can promote the body's absorption of calories and fat storage, thus inducing obesity, and increasing blood lipids.

Changes in intestinal microflora can increase intestinal permeability, thereby promoting the entry of bacterial serum lipopolysaccharide (LPS) into the blood, and leading to endotoxemia, low-level chronic inflammation in the body, and liver metabolic disorders. Eventually, the body develops metabolic syndromes such as obesity, abnormal glucose metabolism, blood lipid disorders, and hypertension.

Compared with healthy people, obesity patients have significantly higher levels of LPS. As a component of the cell wall of Gram-negative bacteria, LPS is considered an inflammatory trigger that can promote the occurrence and development of systemic inflammation and related metabolic disorders through multiple pathways. Systemic inflammation caused by LPS has been shown to trigger impaired glucose tolerance and insulin resistance, which are closely related to the development of obesity. In addition, LPS triggers intestinal inflammation through the TLR4-NF-κB signaling pathway and destroys the intestinal epithelial barrier. The intestinal epithelial barrier refers to a physical barrier formed by the tight nectins ZO-1 and occludin between intestinal epithelial cells to resist the invasion of pathogens and bacterial products.

Obesity can lead to the occurrence of non-alcoholic fatty liver disease, with symptoms such as hepatocyte vacuolization and inflammatory infiltration, affecting the normal metabolic function of liver. The occurrence of non-alcoholic fatty liver disease is generally accompanied by the occurrence of hepatic insulin resistance. The insulin resistance manifests itself as a glucose metabolism disorder in liver metabolism, that is, the liver is unable to convert excess glucose in the blood into glycogen for storage, and then gradually loses its function of regulating the body's blood sugar concentration. Liver damage caused by oxidative stress is the main culprit leading to liver metabolic disorders and inflammation. Phosphorylation activation of Nrf2 is an important switch that turns on the antioxidant pathways. Furthermore, lipid peroxidation caused by liver oxidative stress can lead to disorders of liver lipid metabolism. Excess lipids that cannot be cleared through normal metabolic pathways may cause lipid accumulation in the liver. In addition, obesity-induced translocation activation of intestinal LPS via the portal vein to the liver may occur, and the levels of related pro-inflammatory factors, such as TNF-α, IL-1β, IL-6, and IFN-γ, can also be significantly increased, and show a mutually reinforcing relationship with oxidative stress in the liver.

Adipose tissue can be divided into white adipose tissue (WAT) and brown adipose tissue (BAT). The WAT is an energy warehouse in vivo, and can store excess energy in the form of fat; while the BAT consumes energy and generates heat through the large number of mitochondria in its cells. Different from the WAT, which accumulates fat, the BAT can burn fat and is therefore considered an important target for the treatment of obesity and obesity-related metabolic disorders. In obesity, the accumulation of WAT leads to an increase in overall body weight. This accumulation of adipose tissue is caused by cell proliferation and hypertrophy. Adipogenesis occurs through adipogenesis and lipogenesis, in which preadipocytes differentiate into mature adipocytes, depending on the expression of C/EBPα, PPARγ, and aP2 proteins in the tissue. Uncoupling protein 1 (UCP1) exists in the mitochondria of BAT and can convert energy generated by the decomposition of glucose and fatty acids into heat energy, inhibiting the production of adenosine triphosphate (ATP, a substance that directly supplies energy to organisms). This mechanism mediates brown fat thermogenesis, thereby helping animals resist hypothermia, obesity, and related metabolic disorders.

Probiotics refer to live bacterial preparations and their metabolites that play a beneficial role by improving the ecological balance of the host's intestinal microflora after being applied, thereby improving the health level and health status of the host. So far, there are three major categories of probiotics that are widely used: Lactobacillus, such as Lactobacillus acidophilus, Lactobacillus casei, Lactobacillus rhamnosus, and Lactobacillus bulgaricus; Bifidobacterium, such as Bifidobacterium longum, Bifidobacterium breve, and Bifidobacterium bifidum; and Streptococcus, such as Streptococcus thermophilus and Streptococcus faecalis. Probiotics are one of the most active sources of research for their anti-obesity benefits. To date, multiple molecular mechanisms underlying the anti-obesity effects of probiotics have been demonstrated as changes in metabolic energy, improvement of the intestinal barrier, modulation of metabolic and immune responses, and modulation of neural activity and appetite. As a result, the exploration of novel probiotics with potential health benefits has attracted great attention.

L. acidophilus is a member of the genus Lactobacillus, family Lactobacteriaceae, order Lactobacillus, class Bacilli, phylum Firmicutes, and the kingdom Mycomonera. This species is a gram-positive bacterium that is anaerobic or facultative anaerobic and non-spore-forming, as well as a bacillus that shows rounded and straight ends and is slender, single, paired or short chain-shaped. This species generally lacks flagella, but can move, and has an optimal growth temperature of 35° C. to 38° C., does not grow at 20° C., and shows poor heat resistance. Folic acid and deoxyribonucleosides are important growth factors required for the growth and reproduction of L. acidophilus, and this species can conduct homofermentation using glucose, fructose, lactose, and sucrose to produce D/L lactic acid, releasing lactic acid, acetic acid, hydrogen peroxide, and some antibiotics. L. acidophilus is the main probiotic not only in the stomach but also in the human small intestine, has strong acid resistance and bile salt resistance, and exhibits strong antagonistic properties against Escherichia coli, Salmonella, and Staphylococcus aureus. L. acidophilus can secrete antibiotic-like substances with broad-spectrum effects and has a high capacity to produce β-galactosidase, and its cell wall peptidoglycan is one of the adhesins of lactic acid bacteria. L. acidophilus exerts antibacterial and antiviral effects by producing a large amount of organic acids, hydrogen peroxide, bacteriocins, or peptides and other substances, and has the effects of regulating immunity, maintaining intestinal microecological balance, and improving body metabolism.

The research on the relevant mechanisms of probiotic strains or related products that have been reported to prevent and/or treat obesity and metabolic syndrome is unclear, and there are problems such as insignificant effects, poor stability, and persistence.

### SUMMARY

In view of the unclear mechanism of action, insignificant effect, and poor stability and durability of probiotic products for treating obesity and metabolic syndrome, the present disclosure provides a product for preventing and/or treating obesity and a metabolic syndrome, and a method of preparation and use thereof.

In a first aspect, the present disclosure provides use of a Lactobacillus acidophilus strain JYLA-126 or a Lactobacillus acidophilus strain JYLA-126 coating solution in preparation of a product for preventing and/or treating obesity or a metabolic syndrome, where the L. acidophilus strain JYLA-126 has been deposited in the China General Microbiological Culture Collection Center (CGMCC), No. 3, 1th courtyard, West Beichen Road, Chaoyang District, Beijing on Jul. 8, 2019, with a deposit number of CGMCC NO. 18095.

In the present disclosure, the product preferably includes a drug, and the L. acidophilus strain JYLA-126 coating solution is preferably prepared by mixing the L. acidophilus strain JYLA-126 with a bacterial coating solution. In the L. acidophilus strain JYLA-126 coating solution, the L. acidophilus strain JYLA-126 has a concentration of preferably 1×10⁴ CFU/mL to 1×10⁹ CFU/mL, more preferably 1×10⁵ CFU/mL to 1×10⁸ CFU/mL, and even more preferably 1×10⁶ CFU/mL to 1×10⁷ CFU/mL. In the present disclosure, a preparation process of the bacterial coating solution includes preferably: completely dissolving gelatin in water to obtain the bacterial coating solution. The gelatin and the water are at a weight-to-volume ratio of preferably 0.1 g:1000 mL. The gelatin is dissolved in the water at preferably 60° C. The water is preferably purified water. The bacterial coating solution coats bacterial cells such that the bacterial cells can be better transported to the intestinal tract to exert therapeutic effects.

In a second aspect, the present disclosure provides a product for preventing and/or treating obesity or a metabolic syndrome, including a L. acidophilus strain JYLA-126 or a L. acidophilus strain JYLA-126 coating solution, where the L. acidophilus strain JYLA-126 and the L. acidophilus strain JYLA-126 coating solution are the same as above and are not described again herein.

In a third aspect, the present disclosure provides a method for preparing the product for preventing and/or treating obesity or a metabolic syndrome, including the following steps: resuscitating the L. acidophilus strain JYLA-126 and resuspending in the bacterial coating solution to obtain the L. acidophilus strain JYLA-126 coating solution.

In the present disclosure, a process of resuscitating the L. acidophilus strain JYLA-126 includes the following steps: inoculating the L. acidophilus strain JYLA-126 into a de Man, Rogosa, and Sharpe (MRS) agar medium, conducting anaerobic culture and aerobic culture in sequence to complete first activation, and then conducting the anaerobic culture and the aerobic culture in sequence to complete second activation to obtain a resuscitated L. acidophilus strain JYLA-126. The first activation and the second activation are conducted at preferably 37° C. A method for preparing the bacterial coating solution is the same as above and is not described again. In the L. acidophilus strain JYLA-126 coating solution of the present disclosure, the L. acidophilus strain JYLA-126 has a concentration of preferably 1×10⁴ CFU/mL to 1×10⁹ CFU/mL, more preferably 1×10⁵ CFU/mL to 1×10⁸ CFU/mL, and even more preferably 1×10⁶ CFU/mL to 1×10⁷ CFU/mL.

In a fourth aspect, the present disclosure provides use of the product in preparation of a product for preventing and/or treating obesity or a metabolic syndrome, where the preventing and/or treating is achieved preferably by regulating intestinal microflora and maintaining intestinal health. More preferably, the preventing and/or treating is achieved by improving liver glucose metabolism and alleviating a liver oxidative stress as well as improving liver lipid metabolism and alleviating a liver inflammation level. More preferably, the preventing and/or treating is achieved by improving white adipose accumulation and promoting brown adipose thermogenesis.

The embodiments of present disclosure has the following beneficial effects:

The present disclosure proposes for the first time that the bacterial cells of the L. acidophilus strain JYLA-126 or L. acidophilus strain JYLA-126 coating solution can be used to prepare products for preventing and/or treating obesity or metabolic syndrome. In addition, the present disclosure provides a product for preventing and/or treating obesity and metabolic syndrome, where the bacterial solution of the L. acidophilus strain JYLA-126 is coated with a bacterial coating solution to better transport the bacterial solution to the intestinal tract to exert therapeutic effects. The L. acidophilus strain JYLA-126 coating solution is administered by gavage for the treatment of obesity mice induced by high-fat diet. Various indicators show that the L. acidophilus strain JYLA-126 coating solution may effectively regulate intestinal microflora and maintain intestinal health. The coating solution also improves liver glucose metabolism and reduces liver oxidative stress, and improves liver lipid metabolism and alleviates the liver inflammation level. Moreover, this coating solution improves white adipose accumulation and promotes brown adipose thermogenesis, thereby achieving effective treatment of the obesity and metabolic syndrome in mice.

In the present disclosure, through the organic combination of microbiology, molecular biology, bioinformatics, and immunology, it is proved that the L. acidophilus strain JYLA-126 has significant advantages in preventing and treating obesity diseases, and helps to promote research on the action mechanism of L. acidophilus strain JYLA-126 in weight loss. The present disclosure creates a weight loss product for obesity patients, which is safe, highly targeted, free of any toxic and side effects, and low in price.

### BRIEF DESCRIPTION OF THE DRAWINGS

To describe the technical solutions in the embodiments of the present disclosure or in the prior art more clearly, the accompanying drawings required for the description of the embodiments or the prior art are briefly describes in the followings. Apparently, a person of ordinary skill in the art may still derive other accompanying drawings from these accompanying drawings without creative efforts.

FIGS. 1A-1J show the growth curve and probiotic property of the L. acidophilus strain JYLA-126, where FIG. 1A show the growth curve; FIG. 1B shows the result of free radical scavenging ability; FIG. 1C and FIG. 1D show the acid-resistance in plating and statistics thereof, respectively; FIG. 1E and FIG. 1F show the bile salt resistance in plating and statistics thereof, respectively; FIG. 1G and FIG. 1H show the results antibacterial test on plates and statistics thereof, respectively; FIG. 1I show the result of cell adhesion experiment; and FIG. 1J show the results of hydrophobicity and aggregation experiments;

FIGS. 2A-2P show the therapeutic effect of the L. acidophilus strain JYLA-126 on high-fat diet-induced obesity in mice, where FIG. 2A shows the body shapes; FIG. 2B, FIG. 2C, and FIG. 2D correspond to the results of food intake, water intake, and energy intake, respectively; FIG. 2E shows the energy intake efficiency; FIG. 2F shows the statistics of weight; FIG. 2G shows the result of liver fat accumulation; FIG. 2H shows the reduction in fat weight and the changes in the weight of different tissues and organs in obesity mice; FIG. 2I shows the LPS content in mouse serum; FIG. 2J shows the serum GLP-1 level; FIG. 2K, FIG. 2L, FIG. 2M, and FIG. 2N correspond to blood glucose levels, insulin levels, HOMA-IR levels, and HOMA-IS levels, respectively; and FIG. 2O and FIG. 2P correspond to glucose tolerance and insulin resistance results, respectively;

FIGS. 3A-3K show the therapeutic effect of the L. acidophilus strain JYLA-126 on intestinal microbial dysbiosis induced by high-fat diet, where FIG. 3A, FIG. 3B, FIG. 3C, FIG. 3D, FIG. 3E, and FIG. 3F show Chao 1 index, Shannon index, and Simpson index, observed species index, goods coverage index, and PD whole tree index results, respectively; FIG. 3G shows the Venn diagram; FIG. 3H shows the NMDS; FIG. 3I shows the PCOA result; FIG. 3J shows the abundance of Bacteroidetes, Firmicutes, and Proteobacteria in the total community; and FIG. 3K shows the influence on the adverse intestinal microflora Acetatifactor, Desulfovibrio, and Parabacteroides;

FIGS. 4A-4N show the effect of L. acidophilus strain JYLA-126 on maintaining intestinal health of obesity mice induced by high-fat diet, where FIG. 4A, FIG. 4B, FIG. 4C, and FIG. 4D show the mouse colon, intestinal permeability, plasma FITC-dextran, and fecal LPS, respectively; FIG. 4E shows the total short-chain fatty acids (SCFAs) content; FIG. 4F shows the hematoxylin and eosin staining (H&E) staining; FIG. 4G shows the result of intestinal inflammation and damage; FIG. 4H shows the result of TLR4-NF-κB signaling pathway; FIG. 4I, FIG. 4J, FIG. 4K, and FIG. 4L show the release of cellular inflammatory factors TNF-α, IL-1β, IL-6, and IFN-γ, respectively; FIG. 4M shows the ZO-1 result; and FIG. 4N shows the occludin protein expression level;

FIGS. 5A-5J shows the effect of L. acidophilus strain JYLA-126 on improving liver glucose metabolism and reducing liver oxidative stress, where FIG. 5A shows the H&E staining and PAS of the liver; FIG. 5B shows the NAFLD score; FIG. 5C shows the phosphorylation expression of IRS-2 and AKT, proteins related to the glycogen synthesis in liver tissue; FIG. 5D shows the expression of antioxidant protein P-Nrf; FIG. 5E, FIG. 5F, and FIG. 5G show the activity of antioxidant enzymes SOD, CAT and GPx in liver tissue, respectively; and FIG. 5H, FIG. 5I, and FIG. 5J show the enzyme activities of liver peroxidation product MDA, liver damage ALT, and AST, respectively;

FIGS. 6A-6L show the effect of L. acidophilus strain JYLA-126 on improving liver lipid metabolism and alleviating liver inflammation level, where FIG. 6A and FIG. 6B show the liver oil red O staining and statistics thereof, respectively; FIG. 6C shows the expression of lipid metabolism proteins centered on P-AMPK; FIG. 6D, FIG. 6E, FIG. 6F, and FIG. 6G show the TG, TC, LDL, and HDL contents in mouse serum, respectively; FIG. 6H shows the LPS content in liver tissue; and FIG. 6I, FIG. 6J, FIG. 6K, and FIG. 6L are the levels of related pro-inflammatory factors TNF-α, IL-1β, IL-6, and IFN-γ, respectively; and

FIGS. 7A-7K show the effect of the L. acidophilus strain JYLA-126 on improving white adipose accumulation and promoting brown adipose thermogenesis, where FIG. 7A and FIG. 7B show the staining and statistics of white adipose tissue (WAT), respectively; FIG. 7C and FIG. 7D show the staining and statistics of brown adipose tissue (BAT), respectively; FIG. 7E, FIG. 7F, FIG. 7G, and FIG. 7H show the weights of four types of adipose tissue: epididymal fat (Epi-WAT), inguinal fat (Ing-SAT), mesenteric fat (Mes-WAT), and perinephric fat (Per-WAT), respectively; FIG. 7I shows the expression of C/EBPα, PPARγ, and aP2 proteins in tissues; and FIG. 7J and FIG. 7K are UCP1 protein expression and statistics thereof, respectively.

### DETAILED DESCRIPTION OF THE EMBODIMENTS

To enable those skilled in the art to better understand the solutions of the present disclosure, the technical solutions in the embodiments of the present disclosure will be clearly and completely described below in conjunction with the accompanying drawings in the embodiments of the present disclosure. Obviously, the described embodiments are only a part of, not all of, the embodiments of the present disclosure. All other embodiments obtained by those of ordinary skill in the art based on the embodiments of the application without creative efforts should fall within the protection scope of the application.

The L. acidophilus strain JYLA-126 used in the following examples or test examples is collected by the inventor's company. The Lactobacillus acidophilus strain JYLA-126 is derived from the feces of a centenarian in Zhejiang Province, China. After screening, isolation, and identification, the gene sequence of this strain is shown as follows:

(SEQ ID NO: 1)
TGCAGTCGAGCGAGCTGAACCAACAGATTCACTTCGGTGATGACGTTGG
GAACGCGAGCGGCGGATGGGTGAGTAACACGTGGGGAACCTGCCCCATA
GTCTGGGATACCACTTGGAAACAGGTGCTAATACCGGATAAGAAAGCAG
ATCGCATGATCAGCTTATAAAAGGCGGCGTAAGCTGTCGCTATGGGATG
GCCCCGCGGTGCATTAGCTAGTTGGTAGGGTAACGGCCTACCAAGGCAA
TGATGCATAGCCGAGTTGAGAGACTGATCGGCCACATTGGGACTGAGAC
ACGGCCCAAACTCCTACGGGAGGCAGCAGTAGGGAATCTTCCACAATGG
ACGAAAGTCTGATGGAGCAACGCCGCGTGAGTGAAGAAGGTTTTCGGAT
CGTAAAGCTCTGTTGTTGGTGAAGAAGGATAGAGGTAGTAACTGGCCTT
TATTTGACGGTAATCAACCAGAAAGTCACGGCTAACTACGTGCCAGCAG
CCGCGGTAATACGTAGGTGGCAAGCGTTGTCCGGATTTATTGGGCGTAA
AGCGAGCGCAGGCGGAAGAATAAGTCTGATGTGAAAGCCCTCGGCTTAA
CCGAGGAACTGCATCGGAAACTGTTTTTCTTGAGTGCAGAAGAGGAGAG
TGGAACTCCATGTGTAGCGGTGGAATGCGTAGATATATGGAAGAACACC
AGTGGCGAAGGCGGCTCTCTGGTCTGCAACTGACGCTGAGGCTCGAAAG
CATGGGTAGCGAACAGGATTAGATACCCTGGTAGTCCATGCCGTAAACG
ATGAGTGCTAAGTGTTGGGAGGTTTCCGCCTCTCAGTGCTGCAGCTAAC
GCATTAAGCACTCCGCCTGGGGAGTACGACCGCAAGGTTGAAACTCAAA
GGAATTGACGGGGGCCCGCACAAGCGGTGGAGCATGTGGTTTAATTCGA
AGCAACGCGAAGAACCTTACCAGGTCTTGACATCTAGTGCAATCCGTAG
AGATACGGAGTTCCCTTCGGGGACACTAAGACAGGTGGTGCATGGCTGT
CGTCAGCTCGTGTCGTGAGATGTTGGGTTAAGTCCCGCAACGAGCGCAA
CCCTTGTCATTAGTTGCCAGCATTAAGTTGGGCACTCTAATGAGACTGC
CGGTGACAAACCGGAGGAAGGTGGGGATGACGTCAAGTCATCATGCCCC
TTATGACCTGGGCTACACACGTGCTACAATGGACAGTACAACGAGGAGC
AAGCCTGCGAAGGCAAGCGAATCTCTTAAAGCTGTTCTCAGTTCGGACT
GCAGTCTGCAACTCGACTGCACGAAGCTGGAATCGCTAGTAATCGCGGA
TCAGCACGCCGCGGTGAATACGTTCCCGGGCCTTGTACACACCGCCCGT
CACACCATGGGAGTCTGCAATGCCCAAAGCCGGTGGCCTAACC

This strain is identified as Lactobacillus, presumably Lactobacillus acidophilus. The Lactobacillus acidophilus strain JYLA-126 has been deposited in the CGMCC, No. 3, 1th courtyard, West Beichen Road, Chaoyang District, Beijing, on Jul. 8, 2019, with a deposit number of CGMCC NO. 18095.

### Example 1 Preparation of Lactobacillus acidophilus Strain JYLA-126 Coating Solution

(1) Resuscitation of Lactobacillus acidophilus strain JYLA-126: the Lactobacillus acidophilus strain JYLA-126 was taken out from bacterium library and inoculated into MRS agar medium, anaerobic culture and aerobic culture were conducted in sequence in a 37° C. constant-temperature incubator to complete first activation, and then second activation was completed to obtain a resuscitated Lactobacillus acidophilus strain JYLA-126.

(2) Preparation of a bacterial coating solution: 0.1 g of gelatin was dissolved in 1,000 mL of purified water completely in a 60° C. water bath to obtain the bacterial coating solution.

(3) The resuscitated Lactobacillus acidophilus strain JYLA-126 was resuspended in the bacterial coating solution, such that the Lactobacillus acidophilus strain JYLA-126 had a concentration of 1×10⁴ CFU/mL to obtain the Lactobacillus acidophilus strain JYLA-126 coating solution.

### Example 2 Preparation of Lactobacillus acidophilus Strain JYLA-126 Coating Solution

(1) Resuscitation of Lactobacillus acidophilus strain JYLA-126: the Lactobacillus acidophilus strain JYLA-126 was taken out from bacterial library and inoculated into MRS agar medium, anaerobic culture and aerobic culture were conducted in sequence in a 37° C. constant-temperature incubator to complete first activation, and then second activation was completed to obtain a resuscitated Lactobacillus acidophilus strain JYLA-126.

(2) Preparation of a bacterial coating solution: 0.1 g of gelatin was dissolved in 1,000 mL of purified water completely in a 60° C. water bath to obtain the bacterial coating solution.

(3) The resuscitated Lactobacillus acidophilus strain JYLA-126 was resuspended in the bacterial coating solution, such that the Lactobacillus acidophilus strain JYLA-126 had a concentration of 1×10⁹ CFU/mL to obtain the Lactobacillus acidophilus strain JYLA-126 coating solution.

### Test Example 1 Growth Curve and Probiotic Study of Lactobacillus acidophilus Strain JYLA-126

1. Growth Curve of Lactobacillus acidophilus Strain JYLA-126

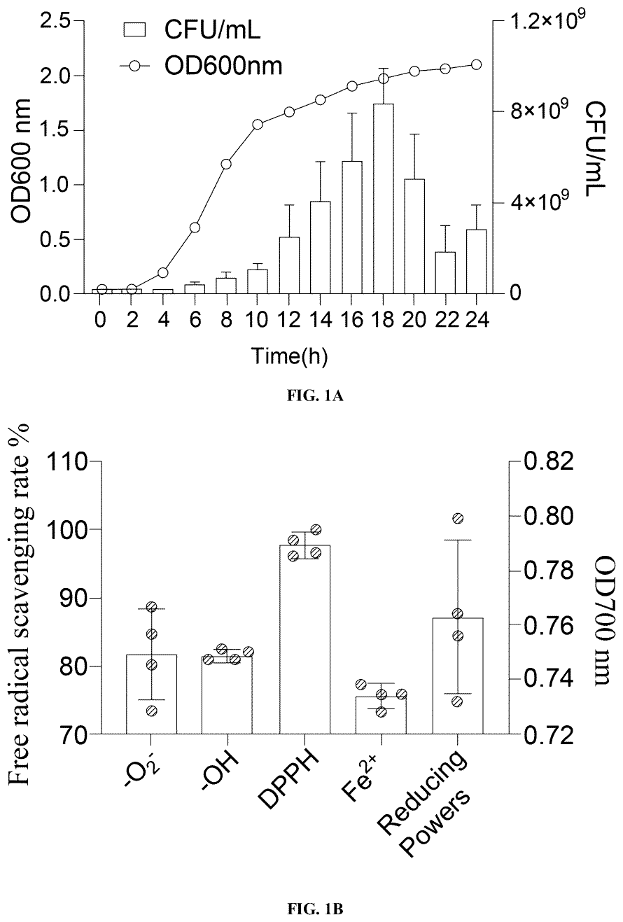

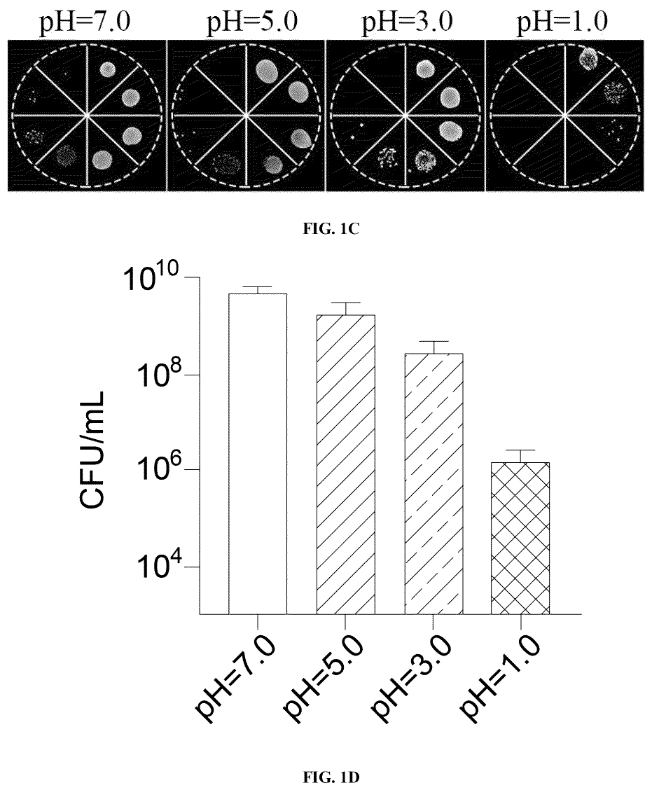

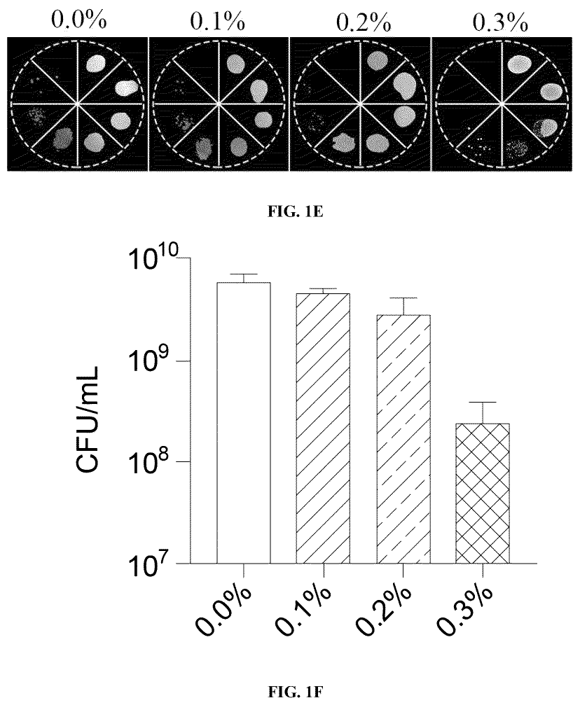

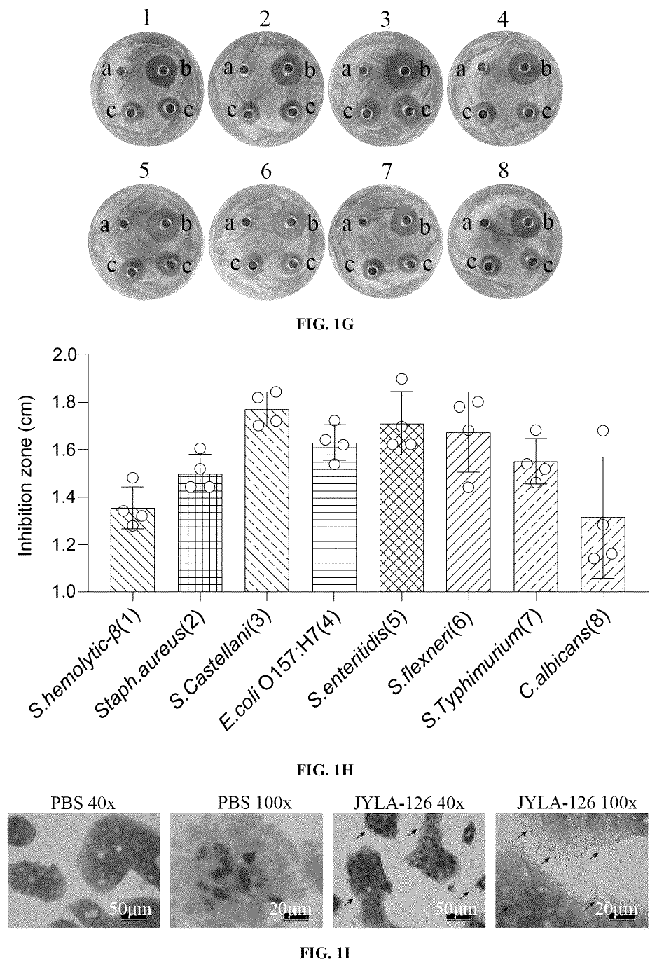

The Lactobacillus acidophilus strain JYLA-126 was taken out from the bacterial library and inoculated into the MRS medium, and was cultured anaerobically in a 37° C. constant-temperature incubator, and then the culture conditions were changed to allow aerobic culture. An optical density (OD) value of the bacterial solution was measured every two hours and measured continuously for 24 h to draw its growth curve, as shown in FIG. 1A. When being cultured at room temperature of 37° C., the Lactobacillus acidophilus strain JYLA-126 entered the logarithmic phase of growth at 4 h and the colony abundance reached its maximum at 18 h, indicating that the bacterium had desirable activity and could be used in subsequent experiments.

2. Study on the Probiotic Properties of Lactobacillus acidophilus Strain JYLA-126

The Lactobacillus acidophilus strain JYLA-126 was cultured in a 37° C. constant-temperature incubator using MRS medium until the OD value was 0.6, then the culture was terminated, and the strain culture solution was retained for later use.

### (1) Determination of Free Radical Scavenging Capacity

a. Determination of Superoxide Radical Scavenging Capacity

2 mL of 150 mmol/L Tris-HCl solution (pH=8.0) was added into 0.5 mL of the strain culture solution, then 1 mL of 1.2 mmol/L pyrogallol solution was added, reacted at room temperature for 30 min, the absorbance at 325 nm was measured, and the superoxide radical scavenging rate was calculated according to the following formula:

$$Superoxide radical scavenging rate = ( 1 - \frac{A 11 - A 10}{A 01 - A 00} \times 100 \%$$

where A00 represents the absorbance without strain culture solution or pyrogallol; A01 represents the absorbance without strain culture solution but with pyrogallol; A10 represents the absorbance with strain culture solution but without pyrogallol; A11 represents the absorbance with strain culture solution and pyrogallol.
   b. Determination of Hydroxyl Radical Scavenging Capacity

A mixed solution containing 1 mL of 2 mmol/L ferrous sulfate solution, 1 mL of 6 mmol/L hydrogen peroxide, and 1 mL of 6 mmol/L salicylic acid was added into 1 mL of the strain culture supernatant, and allowed to stand for 30 min, the absorbance at 510 nm was measured, and with the mixed solution added with 1 mL of distilled water as a reference, the hydroxyl radical scavenging rate was calculated according to the following formula:

$$Hydroxyl radical scavenging rate = ( 1 - \frac{{A}_{1}}{{A}_{0}} ) \times 100 \%$$

where A₁ represents the absorbance of the strain culture supernatant; A₀ represents the absorbance of the distilled water.
   c. Determination of 1,1-Diphenyl-2-Picrylhydrazyl (DPPH) Free Radical Scavenging Capacity

2 mL of the 0.2 mmol/L DPPH methanol solution was added into 2 mL of the strain culture supernatant, and reacted at room temperature for 30 min in the dark, the supernatant was collected to measure the absorbance at 517 nm, and with 2 mL of 0.2 mmol/L DPPH methanol solution added with 2 mL of distilled water as a reference, the DPPH free radical scavenging rate was calculated according to the following formula:

$$DPPH radical scavenging rate = ( 1 - \frac{{A}_{1}}{{A}_{0}} ) \times 100 \%$$

where A₁ represents the absorbance of the strain culture supernatant; and A₀ represents the absorbance the distilled water.

The determination results of the free radical scavenging capacity of Lactobacillus acidophilus strain JYLA-126 were shown in FIG. 1B. The superoxide radical scavenging rate was 81.73%, the hydroxyl radical scavenging rate was 81.50%, the DPPH radical scavenging rate was 97.72%, the ferrous ion chelating rate was 75.52%, and the total reducing power OD value was 0.76.

### (2) Acid and Bile Salt Resistance Test

Acid resistance test: 100 μL of strain culture solution was diluted 10¹, 10³, and 10⁵ times with PBS buffer, centrifuged at 6,000 g for 3 min, the supernatant was discarded, and PBS buffer with pH=2, 3, 4, 5, and 7 was added separately, and allowed to stand for 4 h, mixed well, 10 μL of the sample was taken and spread on a plate, incubated in a constant-temperature incubator at 37° C. for 24 h, the viable bacteria were counted, and the experimental results were recorded. The results are shown in FIG. 1C and FIG. 1D.

Bile salt resistance test: 100 μL of strain culture solution was diluted 10¹, 10³, and 10⁵ times with PBS buffer, centrifuged at 6,000 g for 3 min, the supernatant was discarded, and a medium containing 0%, 0.1%, 0.2%, and 0.3% ox bile salts was added separately, and incubated in a constant-temperature incubator at 37° C. for 12 h, mixed well, 10 μL of the sample was taken and spread on a plate, incubated in a constant-temperature incubator at 37° C. for 24 h, the viable bacteria were counted, and the experimental results were recorded. The results are shown in FIG. 1E and FIG. 1F.

It was shown in the acid and bile salt resistance test that the Lactobacillus acidophilus strain JYLA-126 did not reduce the number of colonies as the acidity of the solution and the concentration of bile salts increased, and there were still an average of 1.57×10⁶ colonies at pH=1.0. When the bile salt concentration was 0.3%, the number of colonies was 2.4×10⁸.

### (3) Bacteriostatic Test:

The Oxford cup method was adopted to test the inhibitory effect against common pathogens. An appropriate amount of pathogen suspension was absorbed with a long cotton swab, excess water was squeezed out, and the pathogens were gently spread on the test plate using a 360° rotation method, the Oxford cup was placed in the corresponding position, then 200 μL of culture solution was absorbed into the Oxford cup, the plate was placed in a 4° C. refrigerator for diffusion for 24 h, and then incubated at 30° C. for 48 h, and the diameter of the inhibition zone was measured and recorded.

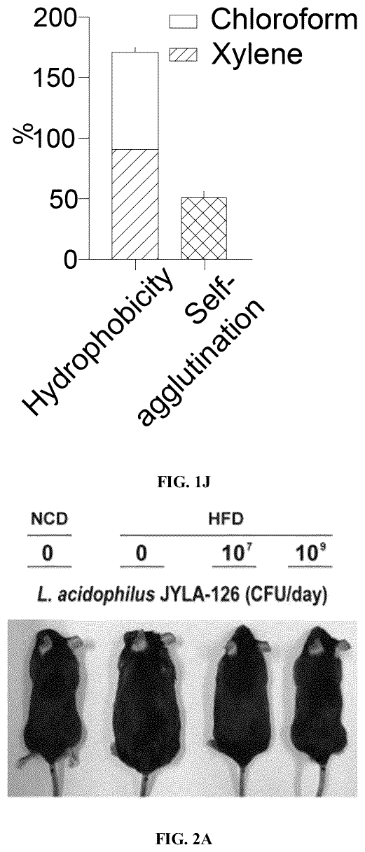

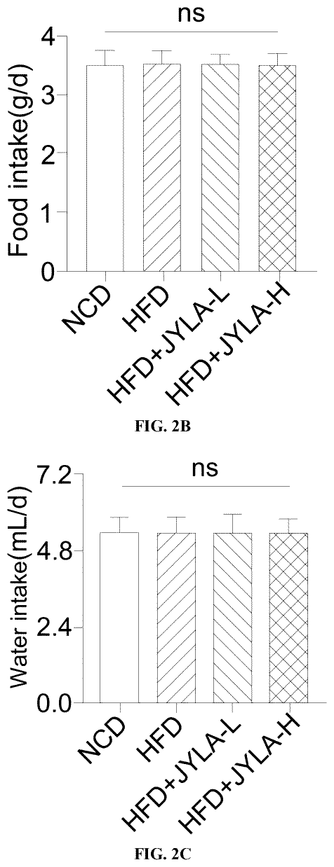

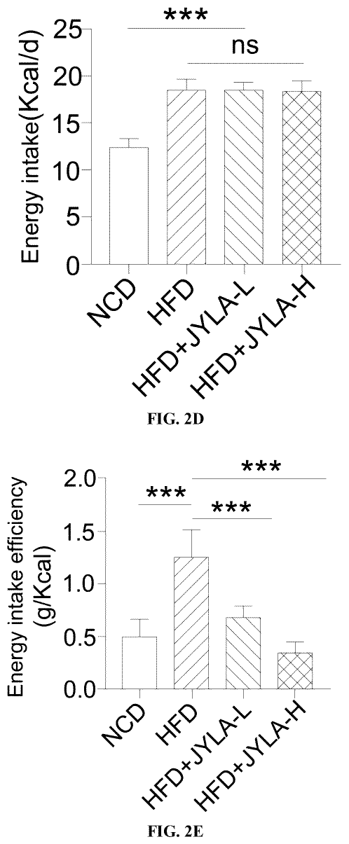

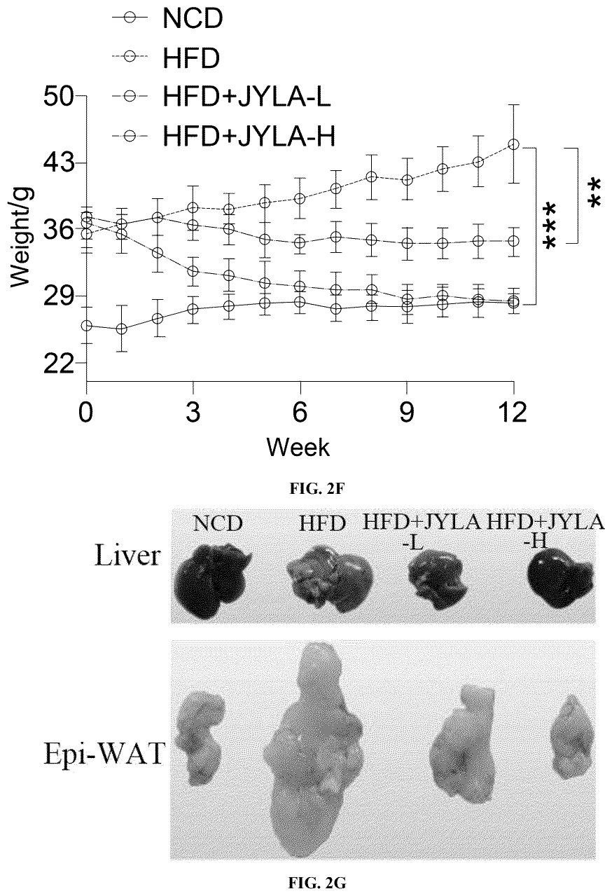

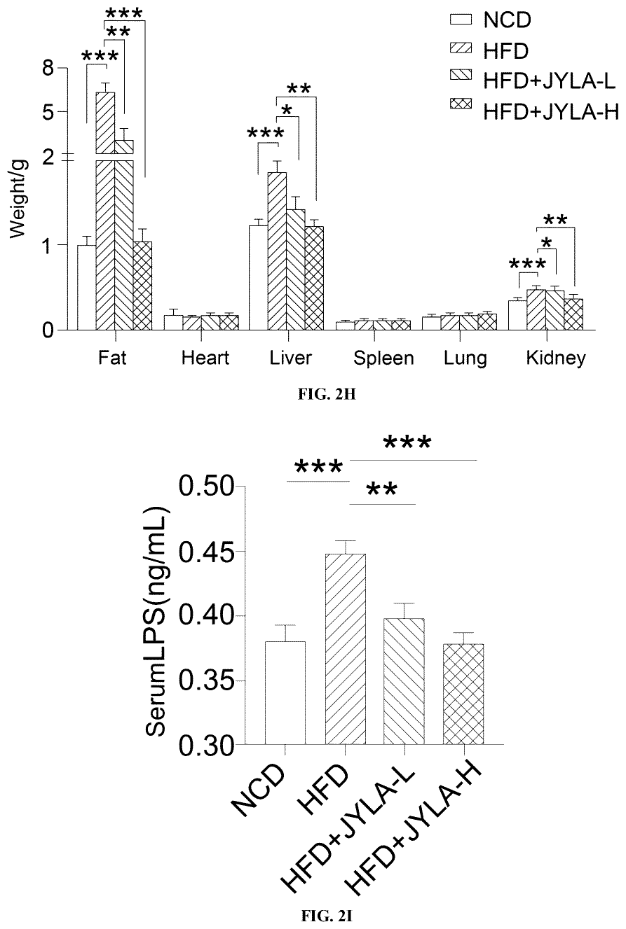

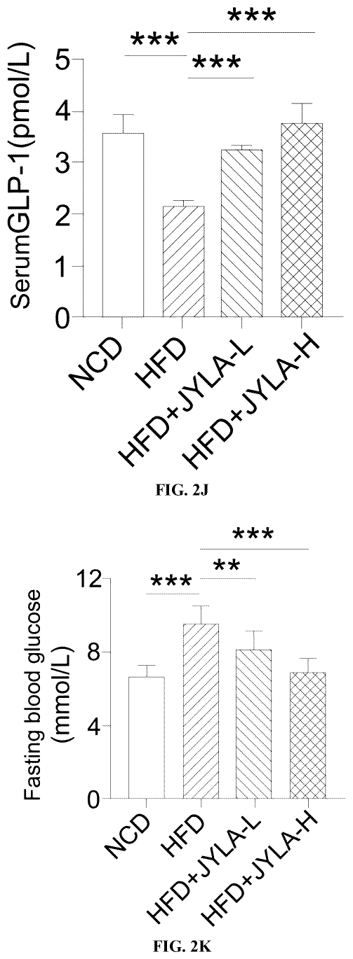

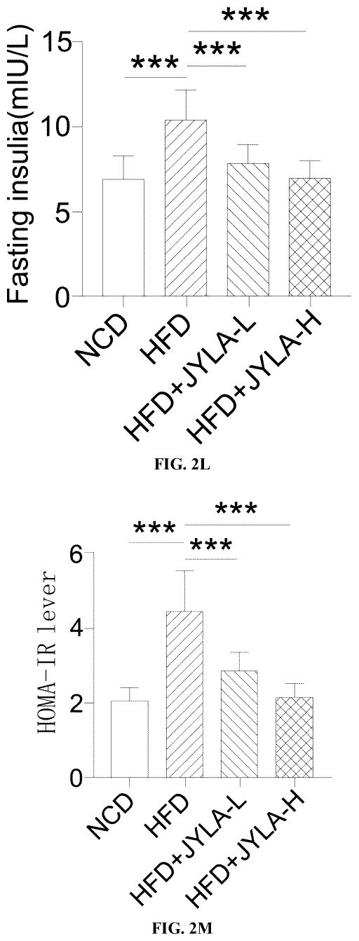

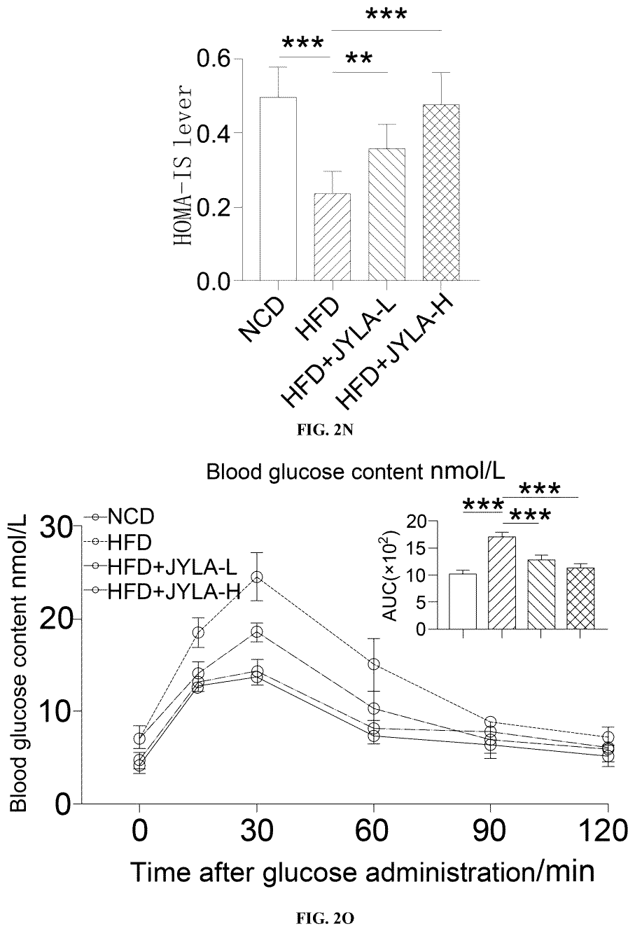

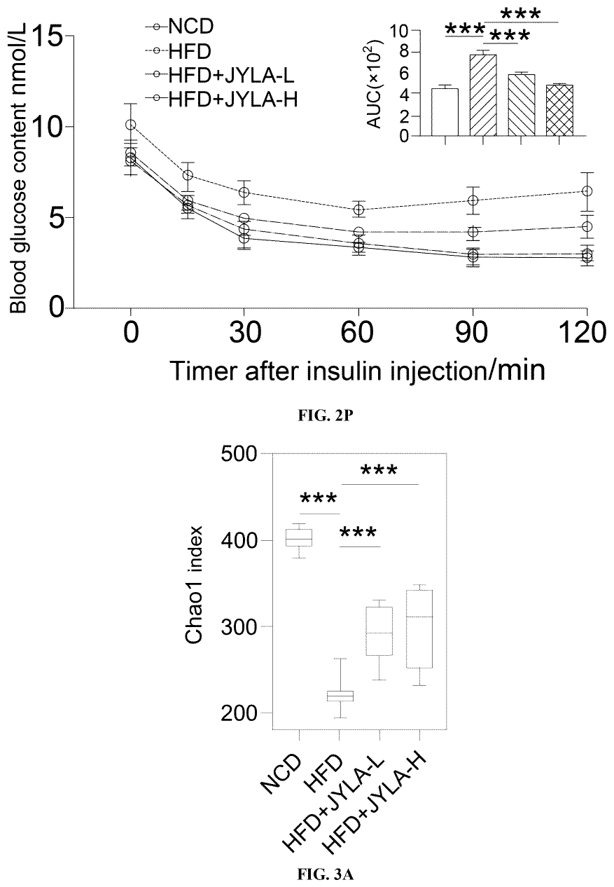

The test results are shown in FIG. 1G and FIG. 1H. The inhibition zones of Lactobacillus acidophilus strain JYLA-126 against β-hemolytic streptococcus (FIG. 1G-1), Staphylococcus aureus (FIG. 1G-2), Shigella Castellani 301 (FIG. 1G-3), Escherichia coli O157:H7 (FIG. 1G-4), Salmonella enteritidis (FIG. 1G-5), Salmonella Typhimurium (FIG. 1G-6), Shigella flexneri (FIG. 1G-7), Candida albicans (FIG. 1G-8) were 1.36 cm, 1.5 cm, 1.77 cm, 1.63 cm, 1.71 cm, 1.68 cm, 1.55 cm, and 1.32 cm, respectively. Compared with the combined antibiotic group, the inhibitory zone was smaller, but compared with the blank control group, there was a larger inhibitory zone.

### (4) Cell Adhesion Experiment

A six-well cell culture plate was washed once with sterile PBS buffer, and placed in a sterilized coverslip. A mixture of 1 mL of strain culture solution and 1 mL of medium containing a small amount of Caco2/HT29 cells was added to the six-well plate, and cultured in a 37° C. cell culture incubator for 1.5 h; the six-well tissue cell culture plate was taken out, the medium was aspirated, and the cells were washed repeatedly with PBS buffer 5 times, fixed with methanol, Gram-stained, observed with an oil microscope, and photographed for documentation. The results, as shown in FIG. 1I, demonstrated that the Lactobacillus acidophilus strain JYLA-126 had desirable adhesion ability to intestinal epithelial cells Caco-2 cells.

### (5) Hydrophobicity and Aggregation Experiments

Three parts of 2 mL of strain culture solution was taken: a first strain culture solution was allowed to stand at room temperature for 3 h, 200 μL of the upper bacterial solution was collected to measure the OD value at 600 nm; a second strain culture solution was thoroughly mixed with an equal volume of xylene for 2 min, and allowed to stand for 30 min to separate the two phases, and the aqueous phase was collected to measure the absorbance value at 600 nm; a third strain culture solution was thoroughly mixed with an equal volume of chloroform for 2 min, and allowed to stand for 30 min to separate the two phases, and the aqueous phase was taken to measure the absorbance value at 600 nm; while physiological saline was used as a blank control. The results were shown in FIG. 1J. The hydrophobicity of Lactobacillus acidophilus strain JYLA-126 in chloroform solvent was 79.87%, the hydrophobicity in xylene solvent was 90.98%, and the self-aggregation rate was 51.49%.

### Test Example 2 In Vivo Animal Experiment

### 1. Establishment of Mouse Obesity Model

After one week of adaptive feeding of 6-8 week old C57BL/6J male mice, the normal maintenance feed of the mice was replaced with a high-fat diet (60% fat). Healthy mice were weighed before feeding, and then fed a pre-determined amount of high-fat diet. Their body weight was monitored weekly, and their blood sugar was tested once a week. The mice were tested for glucose tolerance and insulin tolerance at the 10th and 11th weeks, respectively, and then fed for another week. A curve was plotted based on the previous weekly weight and the mouse obesity degree was calculated (using the formula shown below). When the mouse obesity degree exceeded 20%, the model was considered established.

$$Obesity degree ( \% ) = \frac{Average weight of experimental group - Average weight of control group}{Average weight of control group} \times 100$$

### 2. Treatment of Obesity Mice

12 healthy mice and 36 obesity model mice were taken, and the 36 obesity model mice were randomly divided into 3 groups, with 12 mice in each group. All mice were fed according to the following requirements. The bacterial coating solution was prepared by adding 1,000 mL of purified water with 0.1 g of gelatin in a 60° C. water bath until the gelatin was completely dissolved, and stored at room temperature. The drinking water of mice in each group was changed every two days for about 12 weeks.
(i) Blank control group (normal control diet (NCD)): healthy mice without obesity were administered only 100 μL of bacterial coating solution by gavage;
(ii) obesity model group (HFD): mice were administered only 100 μL of bacterial coating solution by gavage;
(iii) low-dose probiotic treatment group (HFD+JYLA-L): mice were administered the low-concentration L. acidophilus strain JYLA-126 coating solution (1×10⁴ CFU/mL) prepared in Example 1 by gavage;
(iv) high-dose probiotic treatment group (HFD+JYLA-H): mice were administered the high-concentration L. acidophilus strain JYLA-126 coating solution (1×10⁹ CFU/mL) prepared in Example 2 by gavage.

### 3. Phenotypic Indicator Detection and Biochemical Indicator Detection

### (1) Phenotypic Detection Indicators

a. Detection of Weight, Waist Circumference, and Body Length

The weight, waist circumference, and body length of each mouse were measured before the model establishment. After the model establishment, the weight, waist circumference, and body length of each mouse were measured every week.

b. Detection of Food Intake and Water Intake

The weekly food and water intake of each group of mice was recorded for statistics. The effectiveness of each treatment in alleviating the symptoms of polydipsia and polyphagia was tested.

c. Detection of Blood Sugar

After the fasting overnight, blood glucose was measured from the tail vein of the mice, and blood glucose was measured once a week to observe changes in blood glucose values.

d. Detection of Insulin Tolerance and Glucose Tolerance

Insulin tolerance test (ITT): in the sixth week, mice were fasted for 6 h, and then intraperitoneally injected with insulin (0.75 U/kg), and blood samples were collected from the tail vein at different time points (0, 15 min, 30 min, 60 min, 90 min, and 120 min) after insulin administration to detect blood glucose concentrations.

Glucose tolerance test (GTT): in the seventh week, mice were fasted for 12 h, and then intraperitoneally injected with glucose (2 g/kg), and blood samples were collected from the tail vein at different time points (0, 15 min, 30 min, 60 min, 90 min, and 120 min) after glucose administration to detect blood glucose concentrations.

The homeostatic model assessment for insulin resistance (HOMA-IR) index was as follows:

$$HOMA - IR = \frac{Fasting insulin ( µ UI / mL ) \times Fasting glucose ( mM )}{22.5}$$

### (2) Detection of Biochemical Indicators

a. Blood Indicators

At the end of the sixth week, mice were fasted overnight. On the next day, about 1 mL of blood was taken from the orbit, centrifuged at 3,500 rpm for 15 min, and the upper serum sample was taken and frozen. In addition, 500 μL of blood was taken from the heart and placed in heparin-containing anticoagulant tubes for the test of blood routine and biochemical index, including aspartate aminotransferase activity (AST), alanine aminotransferase activity (ALT, to evaluate liver damage), total cholesterol (TC), triglyceride (TG), low-density lipoprotein (LDL), high-density lipoprotein (HDL); malondialdehyde (MDA), catalase (CAT), superoxide dismutase (SOD) and reduced glutathione (GSH) in serum were determined; enzyme-linked immunosorbent assay (ELISA) was conducted to detect plasma insulin and plasma metabolic endotoxin (LPS).

b. Fat Indicators

Brown, subcutaneous, and visceral fat samples were carefully collected along the interscapular region, gastrocnemius muscle, liver, intestine, pancreas, and heart of the mouse. The visceral fat includes perigastrointestinal fat, perirenal fat, periepididymal fat, and retroperitoneal fat. The subcutaneous fat tissue is distributed under the skin and had large fat cells, large lipid droplets, and few organelles, while the opposite is true for visceral fat.

WAT: this type of fat can convert sugars and lipids that the human body cannot consume temporarily into triglycerides and store them, and is mainly related to energy storage. WAT mainly includes the groin, gonads, and subcutaneous fat.

BAT: this type of fat burns excess energy in the body and reduces the energy accumulated in the form of fat in the body, and is generally referred to as “good fat”. BAT is filled with a large number of mitochondria inside, and looks brown. In the kidneys, adrenal glands, peri-aorta, mediastinum and neck tissues, the BAT is best and most obvious in the scapular area.

The mRNA expression of inflammatory cytokines IL1β, IL-2, IL6, TNF-α, RANTES (activating and regulating the expression and differentiation of normal T cells), MCP-1 (monocyte chemoattractant protein), and leptin and other adipokine were detected by quantitative polymerase chain reaction (Q-PCR).

ACC (acetyl-CoA carboxylase), FAS (fatty acid synthase), and UCP1 (uncoupling protein in brown fat) in WAT were detected by Western-blot.

c. Liver Indicators

After the treatment, the mice were anesthetized; and after dissection, the pancreas, liver, spleen, kidneys, intestines, muscles and other organs were removed. As much tissue as possible was taken and frozen for subsequent experiments. The organ index was calculated, and the tissues were frozen in liquid nitrogen and stored in paraformaldehyde for future use. Liver index=average liver weight (mg)/average mouse weight (g).

After dissection, photos of liver size were taken, cell morphology and size were observed with H&E staining, glycogen distribution was observed with liver Periodic Acid-Schiff stain (PAS), and oil droplet accumulation was observed with oil red O staining.

Lipid related: AST, ALT (evaluation of liver damage), TC, TG, LDL, and HDL.

Oxidative stress: MDA, CAT, SOD, and GSH.

Western-blot detection showed that the AMPK signaling pathway was related to lipid metabolism (AMPK, ACC, PPAR-r); the Nrf-2 signaling pathway was related to oxidative stress (Nrf-2, CYP2E1); NFkB signaling pathway (TLR4, P65, IkBα, COX2) was related to inflammation.

d. Indicators of intestine and its content

Before sacrificed, the mice were fasted overnight. After dissection, the duodenum, ileum, cecum, colon, and their corresponding contents were taken and frozen for high-throughput sequencing or protein expression detection.

Colon: intestinal barrier function-related proteins occludin, E-cadherin, and intestinal permeability protein Zo-1 were detected using Western-blot.

Ileum contents: Q-PCR detection of probiotics-AKK, lactic acid bacteria, bifidobacteria, Bacteroidetes, Firmicutes, and their ratios; harmful bacteria, bacteria that decompose tryptophan and produce hydrogen sulfide (Enterobacter aerogenes, Proteus), indole-producing bacteria (pathogenic Escherichia coli, Edwardsiella tarda, Vibrio cholerae); the number of Enterobacteriaceae, Enterococci, Fusobacteria, Veillonella, and Staphylococcus; yeast, Streptococcus, Bacteroides, Lactobacillus, Peptococcus, and Bifidobacterium.

Cecal contents: the extracted intestinal (feces) microbial genomic DNA was sent to Shanghai PersonalBio for 16SrRNA sequencing. The V3-V4 region of the 16SrRNA gene (primers 338F/806R) was amplified using the extracted genomic DNA as a template, and the operational taxonomic units (OTUs) of each sample were obtained by on-board sequencing, and then the correlation between intestinal microflora and obesity was analyzed.

f. Feces Indicators

Before and after successful modeling, 2 to 3 pellets of feces from each mouse were taken at the same time every week and at the point when blood sugar dropped, and frozen for use in high-throughput sequencing analysis.

The collected feces were subjected to high-throughput sequencing analysis to detect fecal LPS content and total short-chain fatty acid content. The total content of short-chain fatty acids was detected using an ELISA kit for rapid detection as follows. Mouse feces (>50 mg) were collected, aliquoted, and frozen at −80° C. while avoiding repeated freezing and thawing. During detection, the fecal sample was thawed and added into an appropriate amount of physiological saline and stirred evenly, centrifuged at 3,000 rpm for 5-10 min to take the supernatant, where the whole process was conducted carefully according to the instructions of the ELISA kit.

### 4. Analysis of Detection Results

(1) L. acidophilus Strain JYLA-126 Treats High-Fat Diet (HFD)-Induced Obesity in Mice

As shown in FIG. 2, after intragastric treatment with L. acidophilus strain JYLA-126 coating solution at different concentrations, it was clearly observed that compared with the bloated bodies of obesity mice, mice in the normal diet mice and mice in the high-dose L. acidophilus strain JYLA-126 treatment group have slender bodies, with greatly reduced body fat content. There were great differences in the body shapes of each group (FIG. 2A). In addition, it was observed that L. acidophilus strain JYLA-126 could effectively inhibit HFD-induced weight gain (FIG. 2F). However, there were no significant differences in food intake, water intake, and energy intake between the HFD group, HFD+JYLA-L group, and HFD+JYLA-H group (FIG. 2B-FIG. 2D). This indicated that the anti-obesity effect of L. acidophilus strain JYLA-126 did not come from a reduction in food intake or energy intake, and the energy intake efficiency of obesity mice was significantly reduced after treatment with L. acidophilus strain JYLA-126 (FIG. 2E). This was further confirmed by the decrease in fat weight and changes in the weight of different tissues and organs in obesity mice (FIG. 2H). Morphological observation of liver and epididymal fat tissue showed that L. acidophilus strain JYLA-126 could significantly inhibit HFD-induced liver and fat accumulation (FIG. 2G), indicating that L. acidophilus strain JYLA-126 could help prevent complications caused by obesity, such as non-alcoholic fatty liver disease.

At the same time, the results of this study indicated that serum LPS levels in HFD-fed mice were significantly increased, and treatment with L. acidophilus strain JYLA-126 could effectively alleviate the increase in serum LPS levels in mice (FIG. 2I). In addition, systemic inflammation caused by LPS has been shown to cause impaired glucose tolerance and insulin resistance, which are closely related to the development of obesity. As one of the most important intestinal hormones, glucagon-like peptide 1 (GLP-1) can stimulate glucose-dependent insulin secretion, thereby improving related metabolic syndromes such as obesity and type 2 diabetes mellitus. In this study, HFD significantly reduced serum GLP-1 levels, while treatment with L. acidophilus strain JYLA-126 effectively alleviated this phenomenon (FIG. 2J). In addition, HFD-induced high fasting glucose levels, high insulin levels, high HOMA-IR, and low homeostatic model assessment for insulin sensitivity (HOMA-IS) levels were all effectively reversed by treatment with L. acidophilus strain JYLA-126 (FIG. 2K-FIG. 2N). Moreover, the same results were obtained in glucose tolerance and insulin tolerance experiments in mice. HFD could induce impaired glucose tolerance and insulin resistance in mice. However, oral administration of L. acidophilus strain JYLA-126 could significantly restore the glucose tolerance and insulin resistance of mice (FIG. 2O and FIG. 2P).

(2) L. acidophilus Strain JYLA-126 Alleviates HFD-Induced Intestinal Microbial Dysbiosis

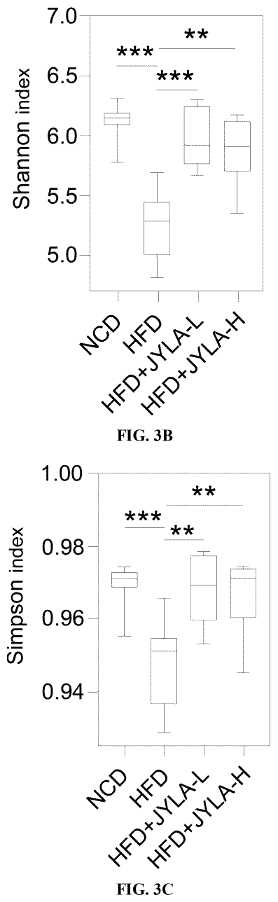

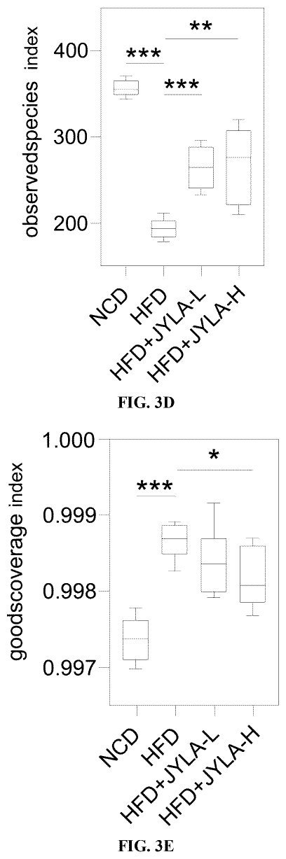

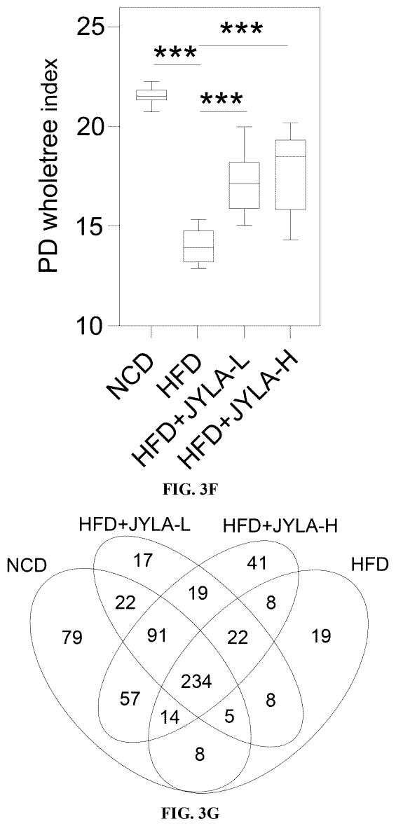

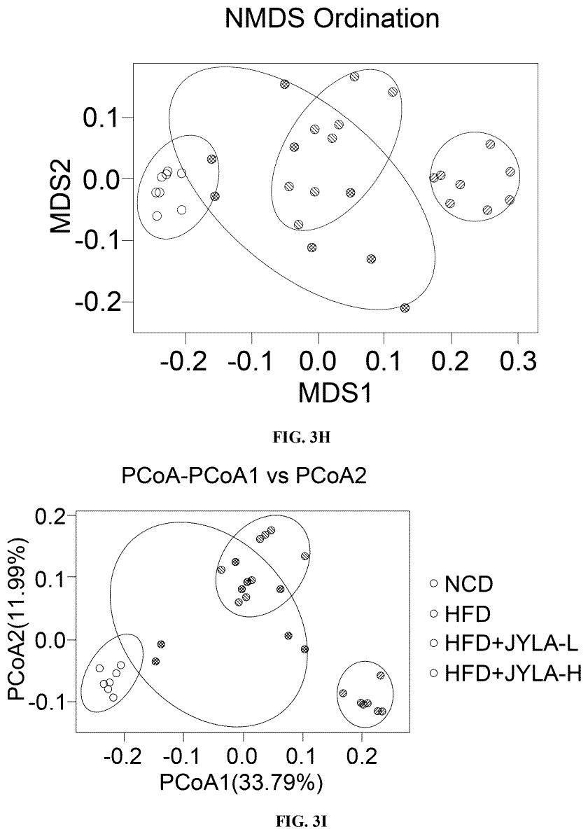

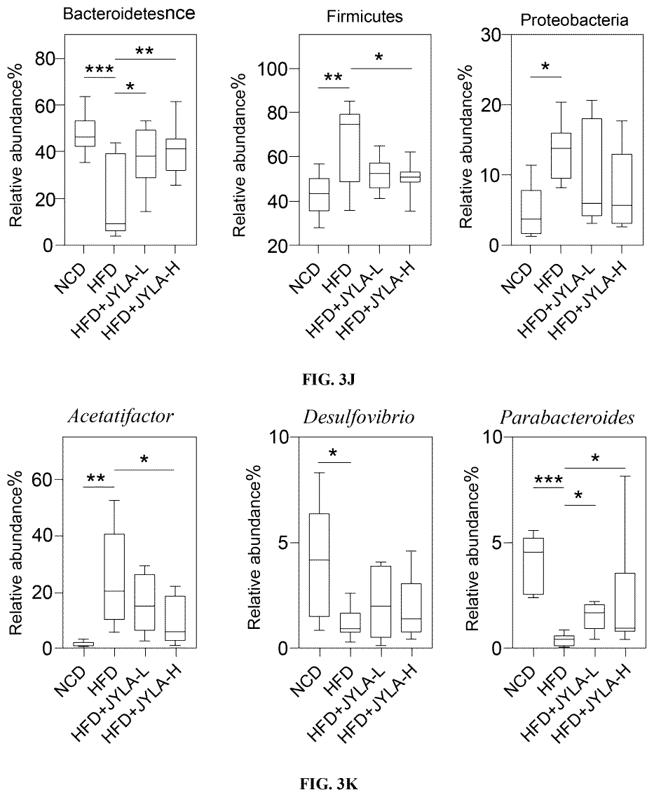

This study applied high-throughput sequencing technology to systematically analyze the changes in the composition of the fecal microbiota of obesity mice after treatment with L. acidophilus strain JYLA-126. The α diversity of intestinal microorganisms shows the richness and evenness of the bacterial microflora, and includes Chao1 index, Shannon index, Simpson index, observed species index, goodscoverage index, and PDwholetree index. HFD feeding significantly reduced a diversity of the intestinal microflora of obesity mice, while supplementation with L. acidophilus strain JYLA-126 completely restored the richness and evenness of the microbial composition (FIG. 3A-FIG. 3F). In order to better understand the similarity in the composition of the intestinal microflora of mice between different groups, a Venn diagram (FIG. 3G) was used to display. Statistics of the number of OTUs in the intestinal microflora of mice in each group showed that the four groups of mice had a total of 644 OTUs, of which 234 OTUs were present in each group at the same time. There were 57 common OTUs between the NCD group and the HFD+JYLA-H group, but there were only 8 common OTUs between the NCD group and the HFD group. In addition, the obesity mice after treatment with the L. acidophilus strain JYLA-126 had 486 OTUs, which was significantly more than the 318 OTUs in the HFD group.

The β diversity of the fecal microbiota of the 4 groups of mice was visually analyzed using non-metric multidimensional scaling (NMDS) and principal coordinate analysis (PCOA). The NMDS results (FIG. 3H) showed that the dots representing the HFD group were all distributed in the right quadrant and had no obvious overlap with the other three groups, indicating that there were significant differences in the fecal microbiota structure between HFD mice and the other three groups. The dots representing the NCD and HFD+JYLA-H groups were mainly distributed in the left quadrant, showing two closely distributed communities. The dots representing the HFD+JYLA-L group only overlapped with the dots of the HFD+JYLA-H group, further confirming that supplementing high concentrations of L. acidophilus strain JYLA-126 could specifically affect the diversity of intestinal microflora and effectively alleviate the dramatic changes in intestinal microflora induced by HFD. The results of PCOA (FIG. 3I) were basically consistent with the analysis results of NMDS.

To further investigate the specific changes in the intestinal microflora community, the relative abundance of dominant phyla and genera in the four groups of microflora was compared. At the phylum level, Bacteroidetes, Firmicutes, and Proteobacteria were the three dominant communities among all taxa, accounting for more than 90% of the total community abundance (FIG. 3J). HFD significantly reduced the relative abundance of Bacteroidetes and increased the relative abundance of Firmicutes and Proteobacteria in the mouse intestine, and treatment with the L. acidophilus strain JYLA-126 could significantly reverse the above changes. At the genus level, the L. acidophilus strain JYLA-126 also showed desirable effects in reversing HFD-induced unfavorable intestinal microflora composition, including a decrease in the genus Acetatifactor and an increase in the genus Desulfovibrio and Parabacteroides (FIG. 3K).

(3) L. acidophilus Strain JYLA-126 Maintains Intestinal Health in Obesity Mice

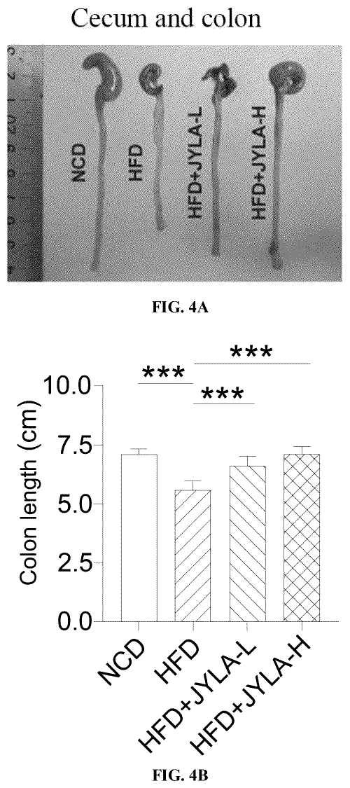

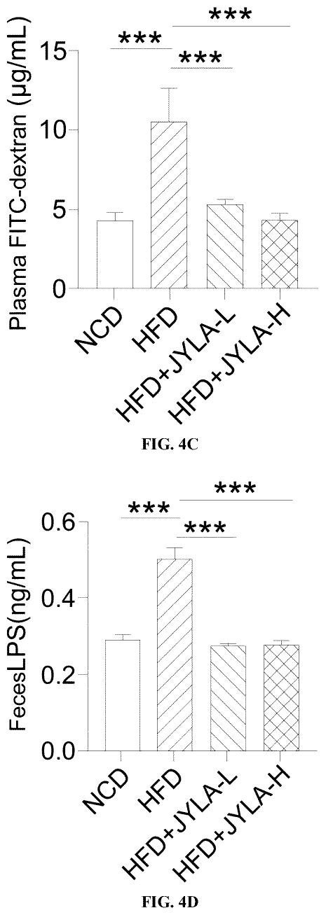

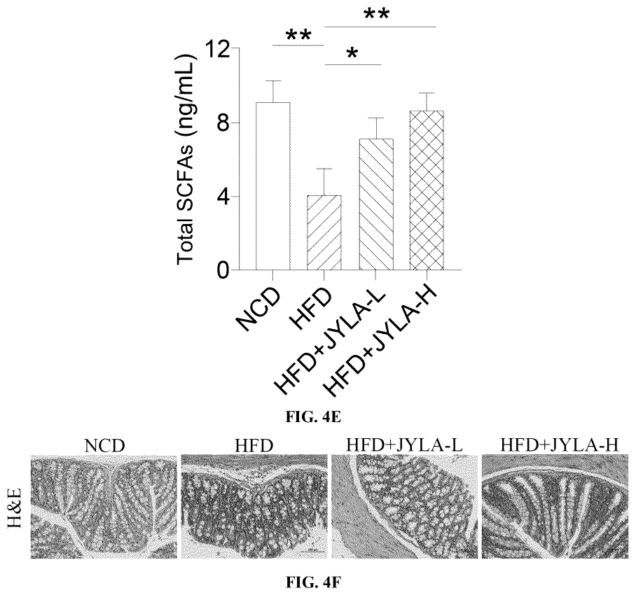

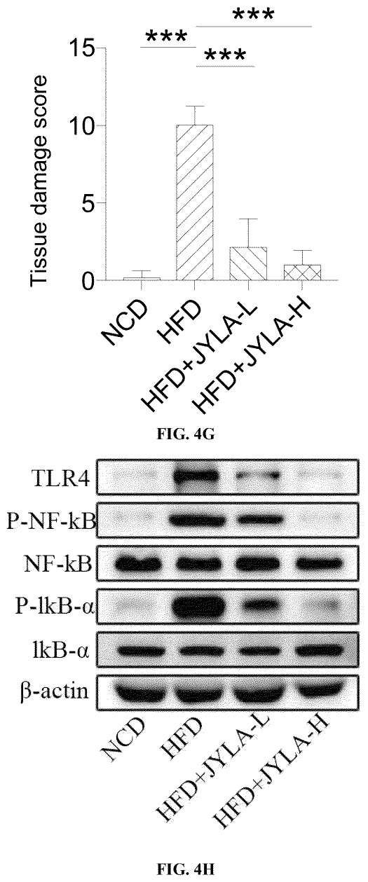

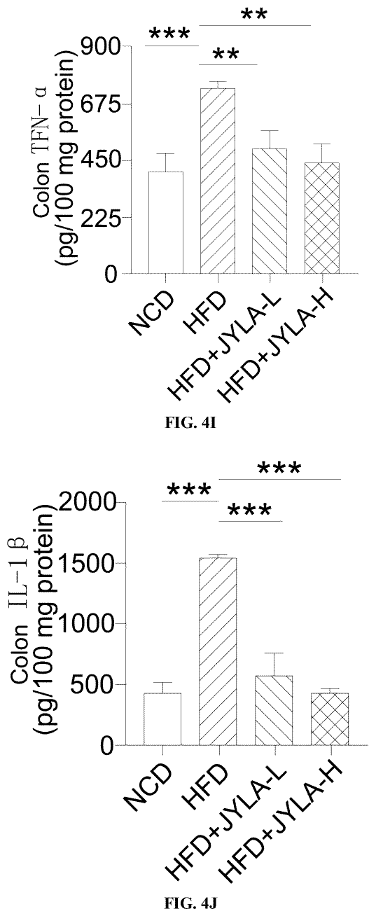

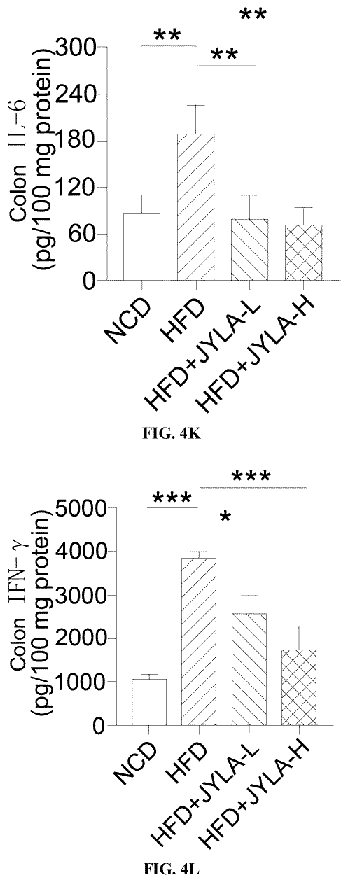

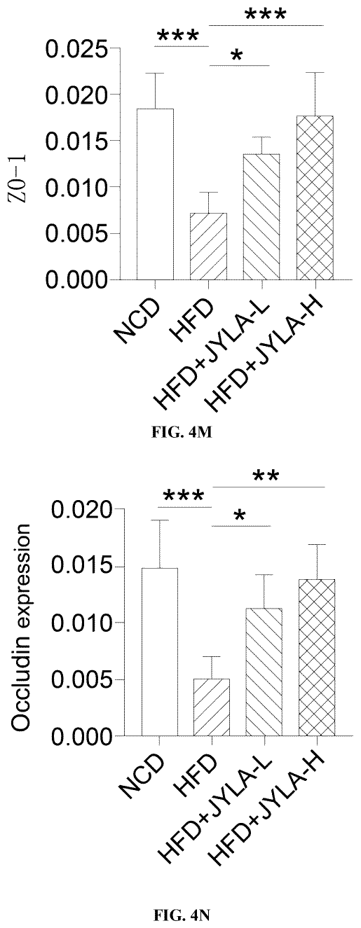

The integrity of the intestinal epithelium is considered the first line of defense in the gastrointestinal tract. HFD can damage the intestinal epithelial barrier by increasing intestinal LPS content. The results of this study consistently showed that HFD-induced mice had colon shortening and significant increases in intestinal permeability (plasma FITC-glucan) and fecal LPS levels (FIG. 4A-FIG. 4D). Hematoxylin and eosin (H&E) staining results confirmed that HFD caused severe intestinal barrier damage, which mainly manifested as significant reduction of intestinal goblet cells, deformation of crypts, infiltration of a large number of inflammatory cells, and thinning of the muscularis mucosa and lamina propria in the HFD group. However, supplementation with the L. acidophilus strain JYLA-126 attenuated HFD-induced intestinal inflammation and damage, accompanied by an increase in total short-chain fatty acids (SCFAs) content (FIG. 4E-FIG. 4G).

The occurrence of intestinal inflammation triggered by LPS mainly depends on the TLR4-NF-κB signaling pathway. The L. acidophilus strain JYLA-126 could reduce the release of LPS and inhibited the activation of TLR-4 by regulating the diversity of intestinal microflora, thereby reducing the phosphorylated expression of downstream NF-κB and IκB-α and reducing the release of cellular inflammatory factors TNF-α, IL-1β, IL-6, and IFN-γ (FIG. 4H-FIG. 4L). The effect of L. acidophilus strain JYLA-126 on the expression levels of mouse colon epithelial tight junction proteins ZO-1 and occludin was detected. Colon immunofluorescence staining results showed that the ZO-1 level of mice in the HFD group was significantly lower than that of mice in the NCD group, while the ZO-1 level of mice in the HFD+JYLA-H group was similar to that of mice in the NCD group. The occludin protein expression levels in each group had the same change trend as that of ZO-1, and there were significant differences (FIG. 4M and FIG. 4N).

(4) L. acidophilus Strain JYLA-126 Improves Liver Glucose Metabolism and Reduces Liver Oxidative Stress

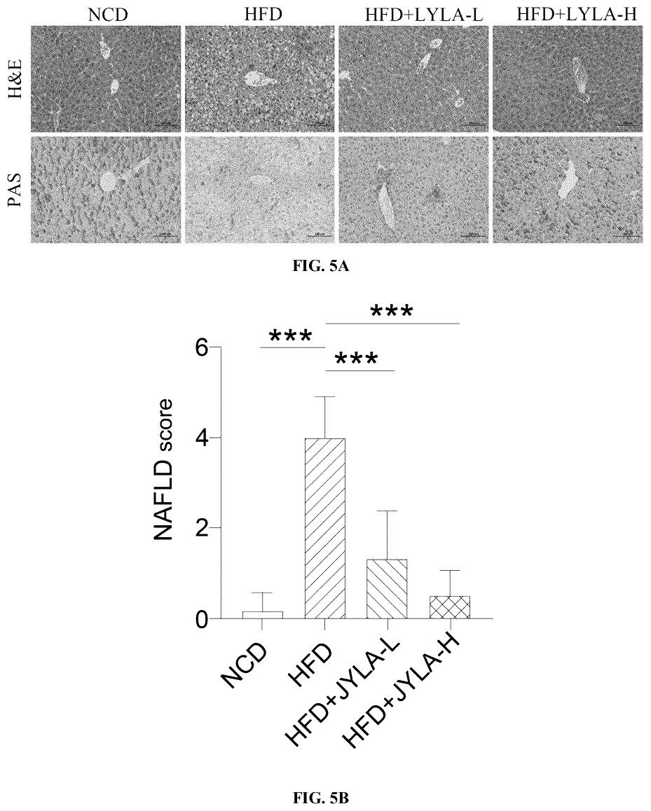

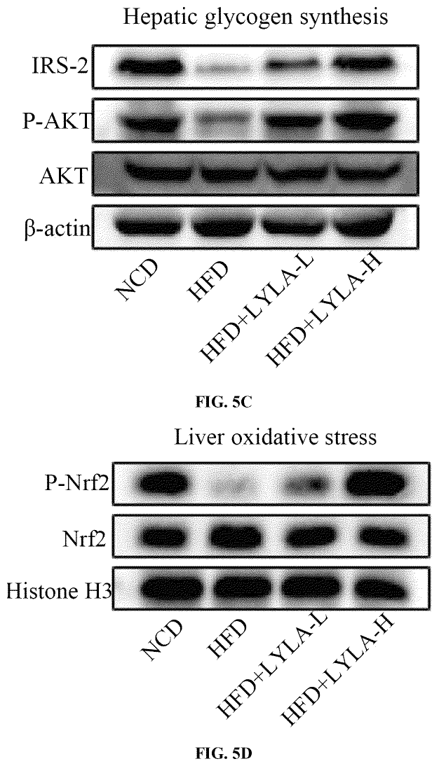

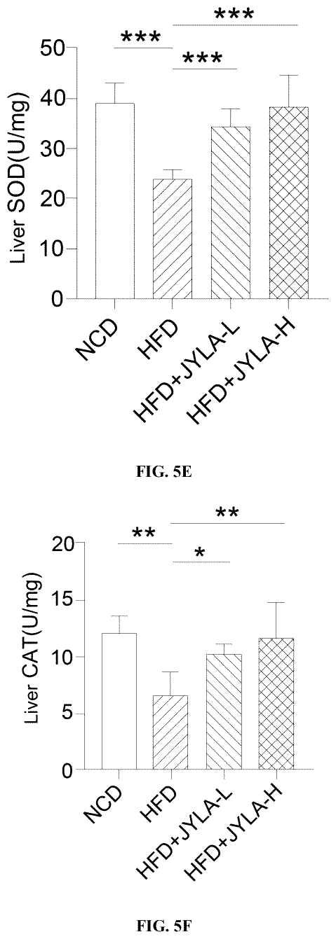

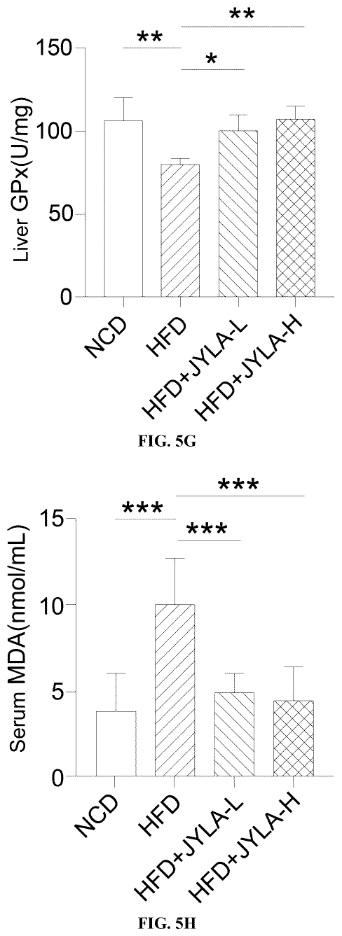

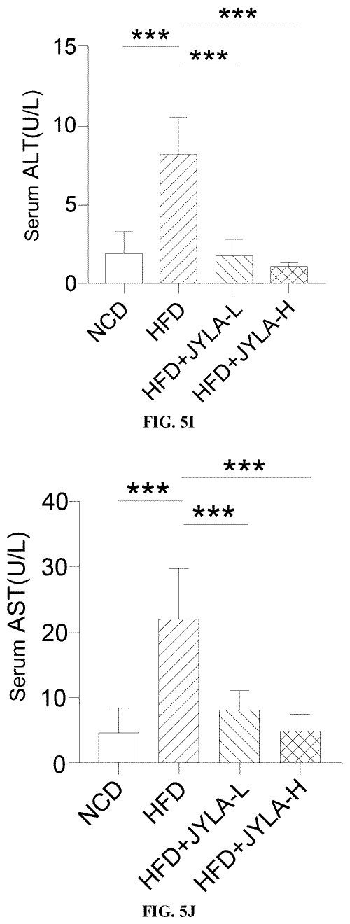

Obesity can lead to the occurrence of non-alcoholic fatty liver disease (NAFLD), which affects the normal metabolic function of the liver. It could be seen from the H&E staining results of the liver that the HFD-induced obesity mice had the highest NAFLD score in the liver and showed more symptoms of fatty liver such as hepatocyte vacuolation and inflammatory infiltration. However, high concentrations of L. acidophilus strain JYLA-126 significantly improved this symptom (FIG. 5A and FIG. 5B). The occurrence of NAFLD is generally accompanied by the occurrence of hepatic insulin resistance. The insulin resistance manifests itself as a glucose metabolism disorder in liver metabolism, that is, the liver is unable to convert excess glucose in the blood into glycogen for storage, and then gradually loses its function of regulating the body's blood glucose concentration. Liver PAS results showed that HFD disrupted the liver's glucose metabolism, resulting in reduced glycogen synthesis. In consistent with the above results, the phosphorylated expression levels of IRS-2 and AKT, proteins related to the glycogen synthesis process, were also significantly reduced in liver tissue. High concentrations of the L. acidophilus strain JYLA-126 could significantly improve this result (FIG. 5A and FIG. 5C).

Studies have shown that liver damage caused by oxidative stress is the main culprit leading to liver metabolic disorders and inflammation. Phosphorylation activation of Nrf2 is an important switch that activates the antioxidant pathway. HFD has induced the expression of antioxidant protein P-Nrf in obesity mice, decreased the activities of antioxidant enzymes SOD, CAT and glutathione peroxidase (GPx) in liver tissue, and increased the content of liver peroxidation product MDA. In addition, liver damage caused by oxidative stress ALT and AST enzyme activities are also increased significantly. In contrast, high concentrations of the L. acidophilus strain JYLA-126 could effectively inhibit the occurrence of oxidative stress in the liver, thus protecting the liver from oxidative damage (FIG. 5D-FIG. 5J).

(5) L. acidophilus Strain JYLA-126 Improves Liver Lipid Metabolism and Alleviates Liver Inflammation Level

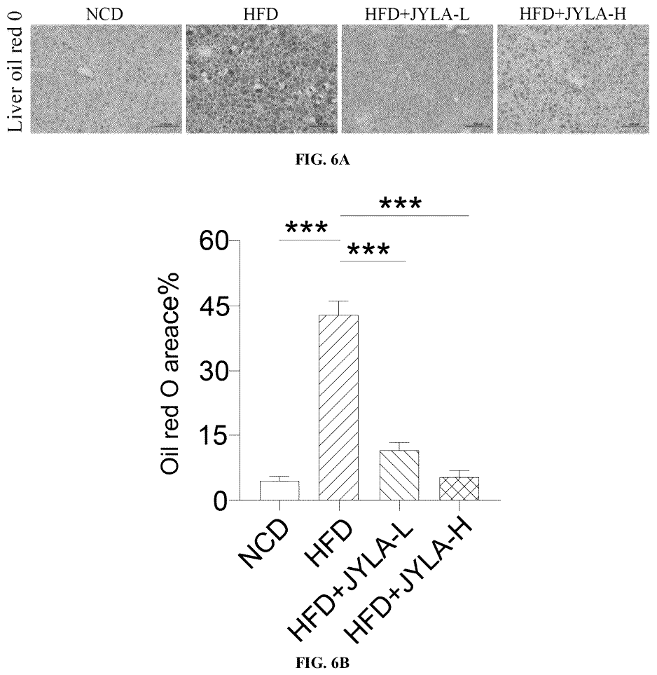

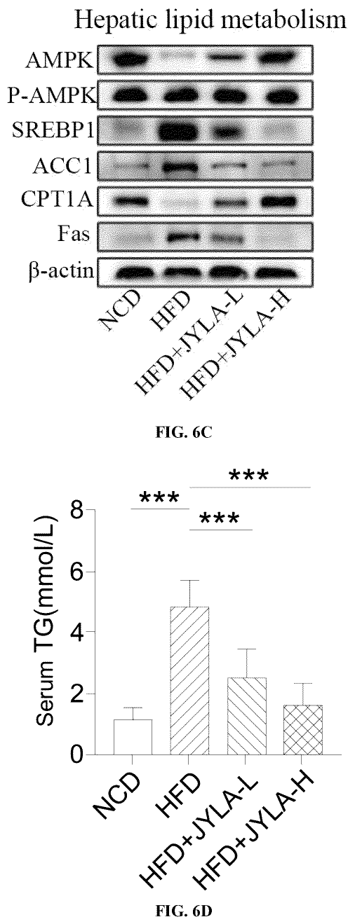

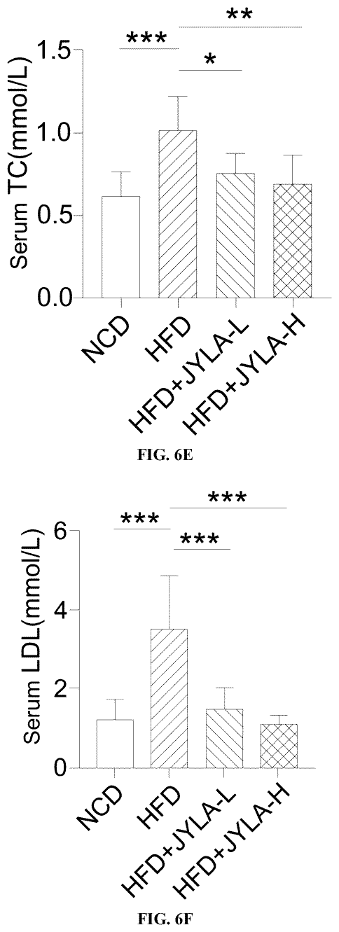

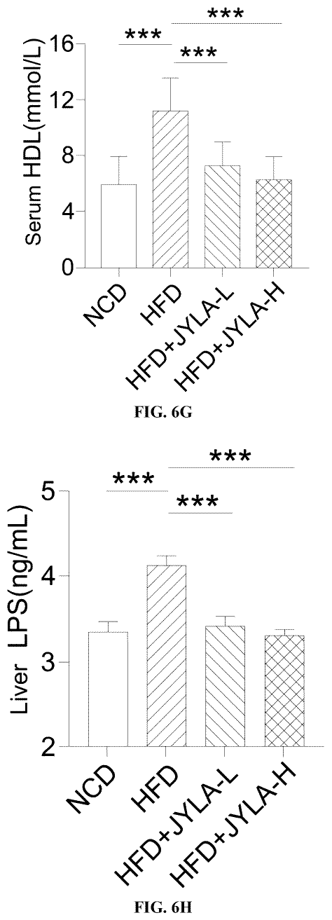

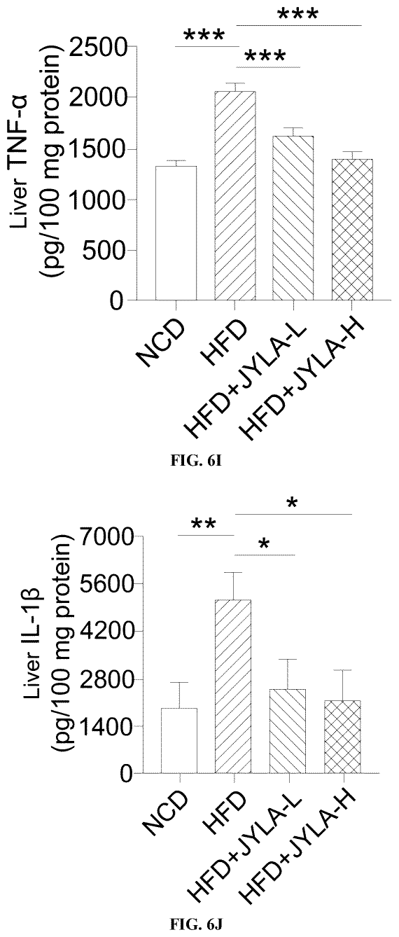

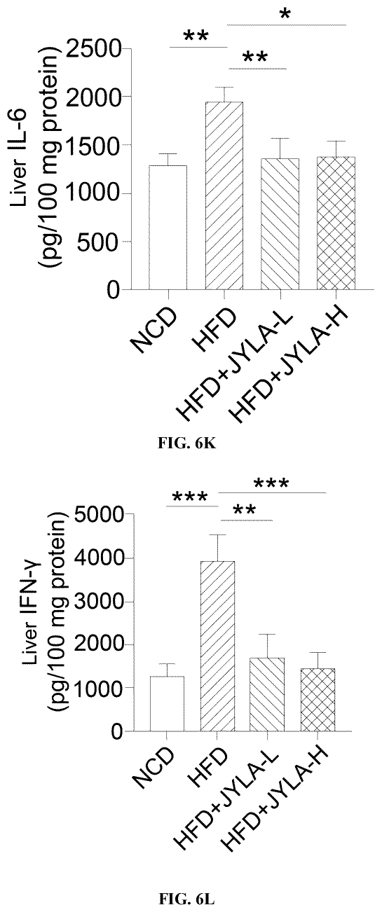

The lipid peroxidation caused by liver oxidative stress can lead to disorders of liver lipid metabolism. Excess lipids that cannot be cleared through normal metabolic pathways may cause lipid accumulation in the liver. The results of oil red O staining of the liver showed that the total area of lipid droplets in the liver of mice in the HFD group significantly increased, and high concentrations of the L. acidophilus strain JYLA-126 could prevent lipid accumulation in the liver (FIG. 6A and FIG. 6B). After investigation into pathways related to lipid metabolism, it is found that HFD can significantly inhibit the expression of lipid metabolism proteins centered on phosphorylated adenosine 5′-monophosphate (AMP)-activated protein kinase (P-AMPK): 1, promoting the expression of SREBP1 and Fas proteins to promote fat synthesis in the liver and increase lipid accumulation; and 2, promoting ACC1 and inhibiting CPT1 protein expression to activate lipid peroxidation. In addition, the blood lipid levels in mouse serum also showed the similar results. The serum levels of TG, TC, LDL and HDL in HFD-induced obesity mice were significantly increased; while mice fed with high concentrations of the L. acidophilus strain JYLA-126 could significantly promote liver lipid metabolism, reduce lipid peroxidation levels, and avoid the occurrence of hyperlipidemia (FIG. 6C-FIG. 6G).

ELISA results showed that the LPS content in the liver tissue of obesity mice significantly increased, and the levels of related pro-inflammatory factors, such as TNF-α, IL-1B, IL-6, and IFN-γ, also increased significantly. This was not only due to the upregulation of inflammation caused by endotoxin LPS, but also had a mutually reinforcing relationship with oxidative stress in the liver. However, high concentrations of the L. acidophilus strain JYLA-126 prevented the infiltration of liver LPS and the expression of pro-inflammatory factors, which was beneficial to liver health and normal metabolism (FIG. 6H-FIG. 6L).

(6) L. acidophilus Strain JYLA-126 Improves White Adipose Accumulation and Promotes Brown Adipose Thermogenesis

Adipose tissue can be divided into white adipose tissue (WAT) and brown adipose tissue (BAT). The WAT is an energy warehouse in vivo, and can store excess energy in the form of fat; while the BAT consumes energy and generates heat through the large number of mitochondria in its cells. Different from the WAT, which accumulates fat, the BAT can burn fat and is therefore considered an important target for the treatment of obesity and obesity-related metabolic diseases.

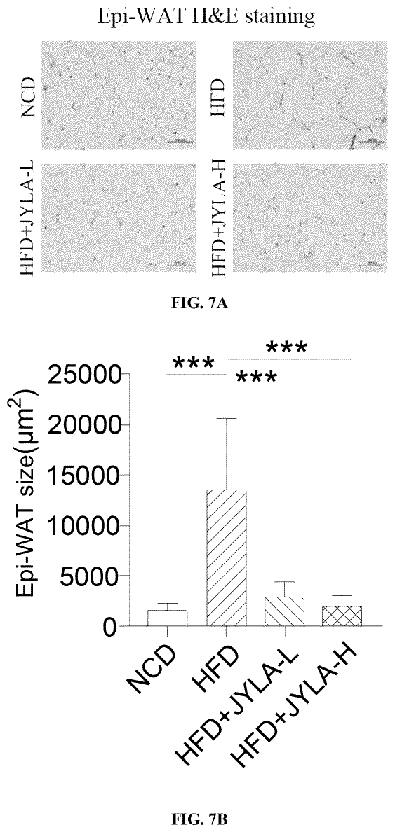

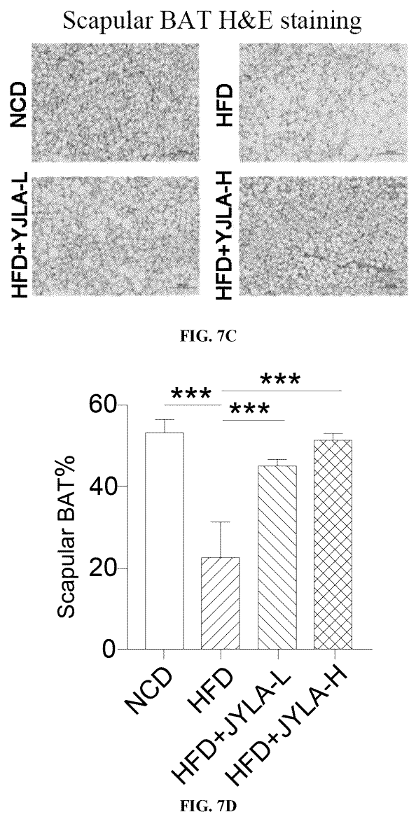

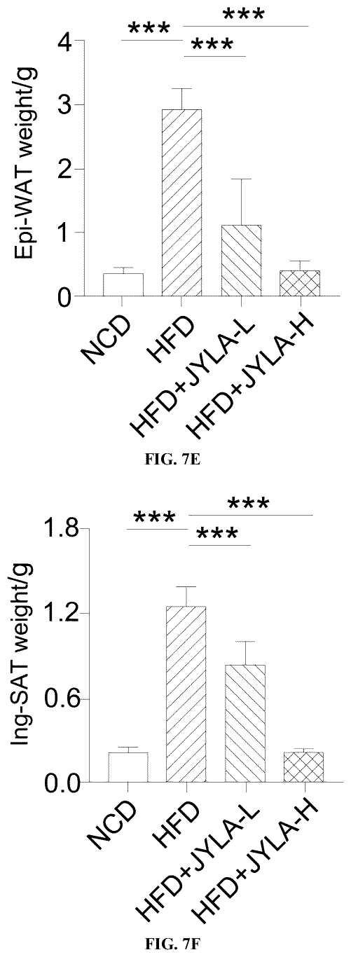

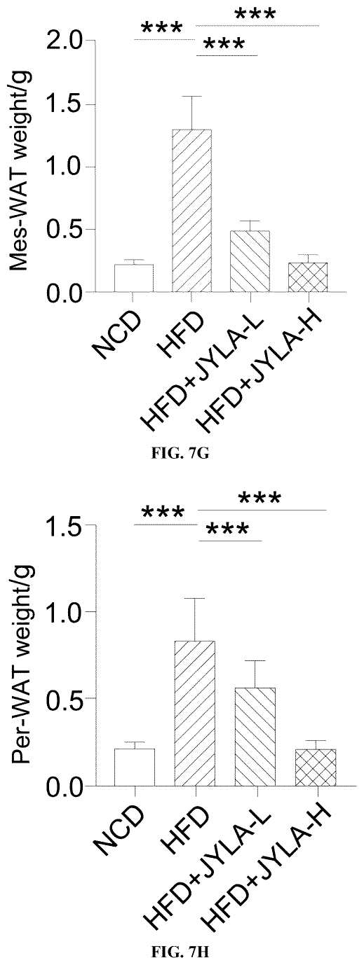

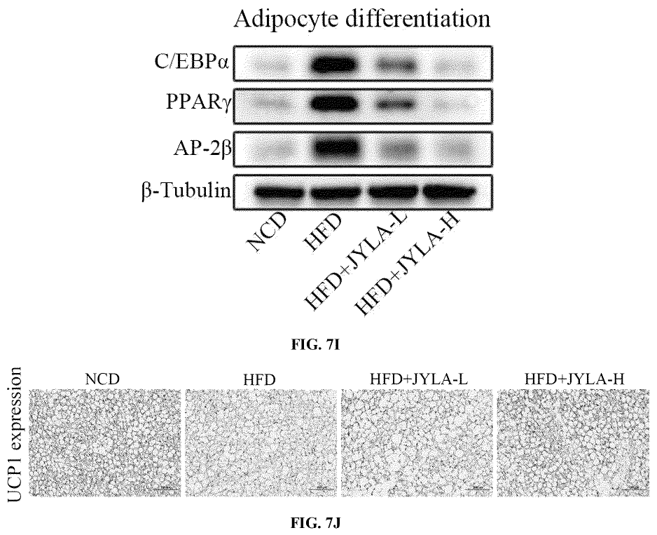

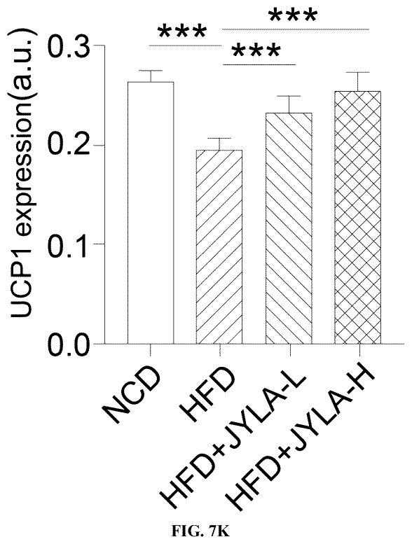

By observation of the H&E staining results of epididymal white adipose (Epididymal WAT), it was found that high concentrations of the L. acidophilus strain JYLA-126 significantly inhibited HFD-induced fat accumulation and adipocyte expansion (FIG. 7A and FIG. 7B). H&E staining results of scapular brown fat (Scapular BAT) showed that the area of BAT cells induced by HFD gradually decreased, and there was a trend of transformation to WAT, that is, the lipid droplets in the cells significantly increased. At the same time, high concentrations of the L. acidophilus strain JYLA-126 prevented the “whitening” of BAT cells (FIG. 7C and FIG. 7D). In addition, as shown in FIG. 7E-FIG. 7H, the weights of four adipose tissues isolated from mice: Epi-WAT, Ing-SAT, Mes-WAT, and Per-WAT were all significantly increased under the action of HFD, while those of L. acidophilus strain JYLA-126 were significantly reduced.

In obesity, the accumulation of WAT leads to an increase in overall body weight. This accumulation of fatty tissue is caused by cell proliferation and hypertrophy, adipogenesis occurs through adipogenesis and lipogenesis, in which preadipocytes differentiate into mature adipocytes, depending on the expression of C/EBPα, PPARγ, and aP2 proteins in the tissue. As shown in FIG. 7I, compared with the HFD treatment group, adding the L. acidophilus strain JYLA-126 could reduce the expression of adipogenic factors C/EBPα, PPARγ, and aP2 proteins in obesity mice.

Uncoupling protein 1 (UCP1) exists in the mitochondria of BAT and can convert energy generated by the decomposition of glucose and fatty acids into heat energy, inhibiting the production of ATP (a substance that directly supplies energy to organisms). This mechanism mediates brown fat thermogenesis, thereby helping animals resist hypothermia, obesity, and related metabolic disorders. As shown in FIG. 7J and FIG. 7K, HFD-fed mice showed inhibition of UCP1 protein expression. In contrast, high concentrations of the L. acidophilus strain JYLA-126 could significantly increase UCP1 expression in obesity mice, thereby promoting the body's energy consumption.

Although the present disclosure has been described in detail with reference to the accompanying drawings and in combination with the preferred embodiments, the present disclosure is not limited thereto. Without departing from the spirit and essence of the present disclosure, those of ordinary skill in the art can make various equivalent modifications or substitutions to the embodiments of the present disclosure, and these modifications or substitutions should fall within the scope of the present disclosure/all changes or substitutions conceived by any person skilled in the art within the technical scope disclosed by the present disclosure should fall within the protection scope of the present disclosure.

1. A method for preventing and/or treating obesity or a metabolic syndrome, comprising administering an therapeutically effective amount of a product comprising a Lactobacillus acidophilus (L. acidophilus) strain JYLA-126 to a subject in need thereof, wherein the L. acidophilus strain JYLA-126 has been deposited in the China General Microbiological Culture Collection Center (CGMCC), No. 3, 1th courtyard, West Beichen Road, Chaoyang District, Beijing on Jul. 8, 2019, with a deposit number of CGMCC NO. 18095.

2. (canceled)

3. The method according to claim 1, wherein the product comprises a drug.

4. The method according to claim 1, wherein the product further comprises a bacterial coating solution.

5. (canceled)

6. A product for preventing and/or treating obesity or a metabolic syndrome, comprising a L. acidophilus strain JYLA-126 coating solution, wherein a L. acidophilus strain JYLA-126 has been deposited in the CGMCC, No. 3, 1th courtyard, West Beichen Road, Chaoyang District, Beijing on Jul. 8, 2019, with a deposit number of CGMCC NO. 1809.

7. The product according to claim 6, wherein the L. acidophilus strain JYLA-126 coating solution further comprises a bacterial coating solution.

8. The product according to claim 6, wherein the L. acidophilus strain JYLA-126 has a concentration of 1×10⁴ CFU/mL to 1×10⁹ CFU/mL.

9. A method for preparing the product according to claim 6, comprising the following steps: resuscitating the L. acidophilus strain JYLA-126 and resuspending in a bacterial coating solution to obtain the L. acidophilus strain JYLA-126 coating solution.

10. The method according to claim 9, wherein the resuscitating comprises the following steps: inoculating the L. acidophilus strain JYLA-126 into a de Man, Rogosa, and Sharpe (MRS) agar medium, conducting anaerobic culture and aerobic culture in sequence to complete first activation, and then conducting the anaerobic culture and the aerobic culture in sequence to complete second activation to obtain a resuscitated L. acidophilus strain JYLA-126.

11. The method according to claim 10, wherein the first activation and the second activation are conducted at 37° C.

12. The method according to claim 9, wherein a preparation process of the bacterial coating solution comprises: completely dissolving gelatin in water to obtain the bacterial coating solution.

13. The method according to claim 12, wherein the gelatin and the water are at a weight-to-volume ratio of 0.1 g:1000 mL.

14. The method according to claim 12, wherein the gelatin is dissolved in the water at 60° C.

15-17. (canceled)


SEQUENCE LISTING (1 sequence)

SEQ ID NO: 1 - DNA, 1415 bp, synthetic construct
  source 1..1415: mol_type = other DNA; note = Gene sequence of Lactobacillus acidophilus strain JYLA 126
tgcagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaacc